(19) United States
(12) Patent Application Publication
(10) Pub. No.: US 2008/0172184 A1
(43) Pub. Date: Jul. 17, 2008

(54) PROTEOMIC PROFILING METHOD USEFUL FOR CONDITION DIAGNOSIS AND MONITORING, COMPOSITION SCREENING, AND THERAPEUTIC MONITORING

(75) Inventors: Jonathan B. Chaires, Louisville, KY (US); Nichola C. Garbett, Louisville, KY (US); A. Bennett Jenson, Louisville, KY (US)

Correspondence Address: STITES & HARBISON, PLLC, 400 W MARKET ST, SUITE 1800, LOUISVILLE, KY, 40202-3352, US

(73) Assignee: UNIVERSITY OF LOUISVILLE RESEARCH FOUNDATION, INC., Louisville, KY (US)

(21) Appl. No.: 11/972,921

(22) Filed: Jan. 11, 2008

Related U.S. Application Data
(60) Provisional application No. 60/978,252, filed on Oct. 8, 2007.
(60) Provisional application No. 60/884,730, filed on Jan. 12, 2007.

(51) Int. Cl. G01N 33/48

(52) U.S. Cl. 702/19

(57) ABSTRACT

A method of diagnosing or monitoring a condition of interest in a subject includes comparing thermograms generated using differential scanning calorimetery. A signature thermogram contains a protein composition pattern for a sample obtained from the subject. The signature thermogram is compared to a standard thermogram. Standard thermograms can include a negative standard thermogram containing a protein composition pattern associated with an absence of the condition of interest, and a positive standard thermogram containing a protein composition pattern associated with a presence of the condition of interest.

### RELATED APPLICATIONS

This application claims priority from U.S. Provisional Application Ser. Nos. 60/978,252 filed Oct. 8, 2007; and 60/884,730 filed Jan. 12, 2007, the entire disclosures of which are incorporated herein by this reference.

### GOVERNMENT INTEREST

Subject matter described herein was made with government support under Grant Number R44 CA103437 awarded by the National Cancer Institute. The government has certain rights in the described subject matter.

### INTRODUCTION AND GENERAL CONSIDERATIONS

The human plasma proteome is a complex fluid that contains over 3000 individual proteins and peptides that are present in quantities that range from picograms to tens of milligrams per milliliter. The expression of specific proteins and specific changes in protein expression levels can be associated with specific conditions, e.g., disease, stage or progression of a condition, infection, etc. As such, analysis of protein levels and changes in protein levels can provide information useful for purposes such as condition diagnosis and therapeutic monitoring. In the clinical setting, certain diagnostic tests include obtaining proteomic profiles of human body fluid collected from a patient. Such diagnostic tests search for protein biomarkers or changes in expression of certain proteins found in body fluids, such as plasma or serum, which can be easily obtained from patients using minimally invasive, safe procedures.

A number of FDA-approved plasma and serum diagnostic assays currently exist; for example, serum and plasma electrophoresis, and a variety of immunochemical assays can be used to monitor the concentrations of specific proteins in plasma and serum. These existing low-to-moderate resolution assays have had a practical impact on medical diagnosis. Such assays can provide useful information at early stages of a disease, allowing for intervention and improved outcomes for patients, with lower associated monetary costs. However, specific protein levels or changes in protein levels associated with conditions of interest can be small, relative to the overall levels of proteins in a given fluid sample. As such, the sensitivity of a method for analyzing protein levels should be such that relatively low levels and minor fluctuations can be detected.

Recent developments in proteomics have brought increased interest in the human plasma and serum proteome as a source for biomarkers of human disease. Higher resolution methods like 2-D electrophoresis and mass spectrometry, coupled with often elaborate protocols for sample preparation and fractionation, have made it possible to identify apparent changes in the composition of the less abundant proteins and peptides in plasma that correlate with particular diseases. Typically no single protein emerges from such analyses as a wholly reliable biomarker, but instead changes in the patterns of panels of proteins often serve as the best diagnostic for a particular malady. These patterns often involve protein or peptide components of plasma that are present in low concentrations.

Interest in the array of existing proteins in a patient's serum has thus evolved to consider in more detail the low molecular weight peptides within serum, which represent a mixture of small intact proteins plus degradation fragments of larger proteins. This low molecular weight region of the serum proteome has been dubbed the “peptidome,” and has been touted as a “treasure trove of diagnostic information that has largely been ignored . . . .” See Liotta and Petricoin, J. Clin. Invest. (2006), and Liotta, et al., Nature (2003). Although some consider the peptidome “unidentified flying peptides” and have questioned the reliability of peptidome SELDI (surface-enhanced laser desorption ionization) patterns as a meaningful diagnostic until the functions of all of the peptide peaks in the peptidome have been properly identified, mass spectrometry, in particular SELDI methods, have made the peptidome accessible for analysis. See Anderson, Proteomics (2005). Many components of the “peptidome” have been found to be complexed with more abundant serum proteins, particular human serum albumin (HAS) and immunoglobulins. Such findings led to the concept of an “interactome,” which introduces the added complexity that serum and plasma can be “comprised of a ‘network’ of protein-protein and peptide-protein interactions,” in which potential biomarkers are bound to the more abundant proteins within the fluid. See Zhou, et al., Electrophoresis (2004). Interestingly, the paper that introduced the “interactome” concept concludes by saying that “the discovery of novel biomarkers in serum/plasma requires new biochemical and analytical approaches, and, most importantly, it is clear that no single sample preparation or detection method will suffice if biomarker investigations are to be broadly successful using current technologies.” See Zhou, et al., Electrophoresis, (2004).

Ten proteins make up 90% of the mass of plasma (by weight). These are, in order of abundance: albumin, IgG, Fibrinogen, Transferrin, IgA, α₂-macroglobulin, α₁-antitrypsin, complement C3, IgM and Haptoglobin. Another 12 proteins account for another 9% of the plasma mass, the 3 most abundant of which are the apolipoproteins A1 and B, and α₁-acid glycoprotein. Twenty-two proteins thus comprise 99% of the mass of plasma, making it challenging to fractionate and quantify the remaining 1%.

The FDA-approved serum protein electrophoresis method monitors changes in the most abundant protein population. See O'Connell, et al., Am. Fam. Physician (2005). However, this method has sensitivity limitations and does not adequately detect changes in less-abundant proteins. Additionally, the equipment necessary for practicing this serum protein electrophoresis method is costly to obtain and maintain.

More recently, 2-D gel electrophoresis and mass spectrometry assays have been developed, which allow for detection of the least abundant components of plasma; however, samples must be prepared by following laborious prefractionation protocols to rid the plasma/serum of the proteins present in high concentrations. See Anderson, Proteomics (2005); Anderson and Anderson, Electrophoresis (1991); Gygi and Aebersold, Curr Opin Chem Biol (2000); Liotta, et al., JAMA (2001); Yates, Trends Genet (2000); and Adkin, et al., Mol Cell Proteomics (2002). Additionally, these assays are time consuming and the equipment necessary for practicing these methods can be costly to obtain and maintain.

Although, the human plasma proteome holds great promise as a convenient specimen for disease diagnosis and therapeutic monitoring, existing assays and technologies have various drawbacks, including sensitivity limitations, time and efficiency limitations, and associated costs that can be prohibitive. Additionally, existing assays and technologies do not fully exploit plasma as a source for biomarkers. For example, electrophoresis and mass spectrometry both separate plasma proteins based on protein size and charge, but assays and technologies based on other physical properties of protein are lacking.

Accordingly, there remains a need in the art for a method for obtaining proteomic profiles of samples, which will address the above-mentioned drawbacks of existing technologies. Additionally, a method with distinctive physical bases, relative to existing technologies, could also be used as an adjunct to existing technologies by identifying unique properties of the individual proteins within a sample.

### SUMMARY

The presently-disclosed subject matter meets some or all of the above-identified needs, as will become evident to those of ordinary skill in the art after a study of the information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a method of diagnosing or monitoring a condition of interest in a subject. In some embodiments, the method includes: generating a signature thermogram containing a protein composition pattern for a sample obtained from the subject; comparing the signature thermogram to a standard thermogram selected from a negative standard thermogram containing a protein composition pattern associated with an absence of the condition of interest, and a positive standard thermogram containing a protein composition pattern associated with a presence of the condition of interest; and identifying the subject as having the condition of interest or lacking the condition of interest.

In some embodiments, the method further includes identifying the subject as having the condition of interest when the signature thermogram is a good simulation of the positive standard thermogram. In some embodiments, the method further includes identifying the subject as having the condition of interest when the signature thermogram is a good simulation of the positive standard thermogram, and the signature thermogram is a poor simulation of the negative standard thermogram.

In some embodiments, the method further includes identifying the subject as lacking the condition of interest when the signature thermogram is a poor simulation of the positive standard thermogram. In some embodiments, the method further includes identifying the subject as lacking the condition of interest when the signature thermogram is a good simulation of the negative standard thermogram. In some embodiments, the method further includes identifying the subject as lacking the condition of interest when the signature thermogram is a poor simulation of the positive standard thermogram, and the signature thermogram is a good simulation of the negative standard thermogram.

In some embodiments of the method, each standard thermogram is a group-specific standard thermogram. In some embodiments, each group-specific standard thermogram is an ethnic group-specific standard thermogram. In some embodiments, each ethnic group-specific standard thermogram is: a Hispanic-specific standard thermogram if the subject is Hispanic; or a non-Hispanic-specific standard thermogram if the subject is non-Hispanic.

In some embodiments, the condition of interest is cancer. In some embodiments, the cancer is selected from: cervical cancer, endometrial cancer, lung cancer, melanoma, multiple myeloma, ovarian cancer, and vulvar cancer.

In some embodiments the condition of interest is a stage of cervical cancer selected from: moderate cervical dysplasia (CIN II), early stage cervical cancer, and stage IVB cervical cancer.

In some embodiments, the condition of interest is an autoimmune disease. In some embodiments, the autoimmune disease is selected from: rheumatoid arthritis, multiple sclerosis, and systemic lupus.

In some embodiments, the condition of interest is caused by a bacterial infection. In some embodiments, the condition is Lyme disease.

In some embodiments, the condition of interest is caused by a viral infection. In some embodiments, the condition is selected from: Dengue fever, and hepatitis.

In some embodiments, the condition of interest is selected from: amyotrophic lateral sclerosis (ALS), anemia, cardiac disease, diabetes, and renal disease.

In some embodiments, the method further includes comparing the signature thermogram to multiple positive standard thermograms, and identifying the subject as having the condition associated with the positive standard thermogram of which the signature thermogram is a good simulation. In some embodiments, the positive standard thermogram is associated with multiple sclerosis, and another of the positive standard thermograms is associated with amyotrophic lateral sclerosis (ALS). In some embodiments, the multiple positive standard thermograms include positive standard thermograms for different stages of a condition of interest.

In some embodiments, the method further includes providing a second sample obtained from the subject at a time point after the first sample is obtained; generating a second signature thermogram containing a protein composition pattern for the second sample; comparing the first signature thermogram to the second signature thermogram; and identifying the condition of interest as changed when the second signature thermogram is a poor simulation of the first signature thermogram, or identifying the condition of interest as being unchanged when the second signature thermogram is a good simulation of the first signature thermogram. In some embodiments the method further includes comparing the second signature thermogram to the negative standard thermogram, and identifying the subject as lacking the condition of interest if the second signature thermogram is a good simulation of the negative standard thermogram. In some embodiments, the method further includes comparing the second signature thermogram to positive standard thermograms for different stages of a condition of interest, and identifying the condition as progressing, unchanged, or regressing in the subject.

The presently-disclosed subject matter includes a method of assessing a treatment program for a subject. In some embodiments, the method includes providing a first sample obtained from the subject at a first time point of interest; generating a first signature thermogram containing a protein composition pattern for the first sample; providing a second sample obtained from the subject at a second time point of interest; generating a second signature thermogram containing a protein composition pattern for the second sample; comparing the first signature thermogram to the second signature thermogram; and identifying the presence or absence of a change in the condition of interest.

In some embodiments, the method further includes identifying the absence of a change in the condition of interest when the second signature thermogram is a good simulation of the first signature thermogram.

In some embodiments, the method further includes identifying the presence of a change in the condition of interest when the second signature thermogram is a poor simulation of the first signature thermogram.

In some embodiments, the first time point of interest occurs prior to the initiation of the treatment program, and the second time point of interest occurs following the initiation of the treatment program. In some embodiments, the method further includes comparing the second signature thermogram to a standard thermogram selected from: a negative standard thermogram containing a protein composition pattern associated with an absence of the condition of interest; and a positive standard thermogram containing a protein composition pattern associated with a presence of the condition of interest.

The presently-disclosed subject matter includes a method of screening for a composition useful for treating a condition of interest. In some embodiments, the method includes administering to a subject infected with the condition of interest a candidate treatment composition; providing a sample obtained from the subject; generating a signature thermogram containing a protein composition pattern for the sample; comparing the signature thermogram to a standard thermogram selected from: a negative standard thermogram containing a protein composition pattern associated with an absence of the condition of interest; and a positive standard thermogram containing a protein composition pattern associated with a presence of the condition of interest; and determining the utility of the candidate treatment composition.

The presently-disclosed subject matter includes a method of screening a composition, e.g. candidate drug or treatment, for plasma protein interactions. In some embodiments, the method includes interacting the composition with a first plasma sample; generating a first signature thermogram containing a protein composition pattern for the first plasma sample; comparing the first signature thermogram to a negative standard thermogram containing a protein composition pattern associated with an absence of plasma protein interactions; or a second signature thermogram generated using a second plasma sample not interacted with the composition; and identifying the composition as lacking substantial plasma protein interactions when the first signature thermogram is a good simulation of the negative standard thermogram, or the second signature thermogram.

### BRIEF DESCRIPTION OF THE DRAWINGS

FIG. 1 is a schematic representation of an exemplary differential scanning calorimeter (DSC), depicting sample (S) and reference (R) cells that are kept in thermal balance by heaters controlled by feedback electronics as both cells are heated at a precisely controlled rate (ΔT₂);

FIG. 2 includes an exemplary thermogram for a two-state denaturation of a protein;

FIG. 3 includes thermograms obtained by DSC, including thermograms for various individual proteins, as well as a thermogram of the weighted sum of the group of individual proteins (solid lines representing 16 individual proteins, and the dashed line for the sum); the individual proteins were weighted according to their actual known concentration in plasma, and the individual proteins were then summed to yield the dashed line, which compares well with actual experimental thermograms of plasma from healthy individuals as shown in FIG. 8;

FIG. 4A includes two superimposed thermograms for “normal” subjects and for subjects suffering from Lyme disease;

FIG. 4B includes the quantile plots obtained after integrating and normalizing the thermograms of FIG. 4A;

FIG. 4C includes the quantile-quantile plot obtained by plotting the normal quantile (x-axis) of FIG. 4B against the Lyme quantile (y-axis) of FIG. 4B;

FIG. 5 is a flow chart illustrating the steps involved in an exemplary method of diagnosing a condition of interest in accordance with the presently-disclosed subject matter;

FIG. 6 is a flow chart illustrating the steps involved in an exemplary method of assessing efficacy of a treatment program in accordance with the presently-disclosed subject matter;

FIG. 7 is a flow chart illustrating the steps involved in an exemplary method of screening for a composition useful for treating a condition of interest in accordance with the presently-disclosed subject matter;

FIG. 8 includes an average thermogram of plasma calculated from samples obtained from 15 normal subjects, where the average thermogram is the black solid line, the standard deviation at each temperature is indicated by the gray shading, and where the vertical dashed line is the first moment of the thermogram;

FIG. 9 includes thermograms for freshly-prepared plasma and serum samples, and thermograms for freeze-thaw plasma and serum samples;

FIG. 10 includes a series of thermograms for the denaturation of individual purified plasma proteins, including α₁-antitrypsin, transferrin, α₁-acid glycoprotein, complement C3, c-reactive protein, haptoglobin, prealbumin, α₂-macroglobulin, complement C4, α₁-antichymotrypsin, IgM, albumin, IgG, fibrinogen, IgA, and ceruloplasmin;

FIG. 11 includes Panel A, showing a series of thermograms (solid lines) for the 16 most abundant plasma proteins, and a calculated thermogram (dashed line) obtained from the sum of the weighted contributions of the 16 most abundant plasma proteins; and Panel B, showing thermograms obtained from mixtures of pure plasma proteins mixed at concentrations that mimic their known average concentrations in normal plasma, where the gray curve is a mixture of HSA, IgG, fibrinogen, and transferrin, and the black curve is a mixture of the 16 most abundant plasma proteins;

FIG. 12 includes thermograms for samples in which albumin was removed from serum, where Panel A shows an expected thermogram (dashed line) based on the weighted sum of the most abundant proteins (solid lines) less HSA and fibrinogen, and where Panel B shows the observed experimental thermogram for albumin-depleted serum, from which HSA was removed by affinity chromatography using a SwellGel Blue™ albumin removal kit;

FIG. 13 includes a series of thermograms, where each panel compares normal plasma with plasma associated with a condition of interest; in Panel A the condition is systemic lupus; in Panel B the condition is Lyme disease; and in Panel C the condition is Rheumatoid arthritis;

FIG. 14 is a bar graph showing the relative concentrations of the major plasma proteins for normal and diseased plasma samples, where concentrations of the individual proteins were normalized with respect to the total protein concentration;

FIG. 15 includes a series of densitometric scans from stained gels for normal samples and samples associated with Rheumatoid arthritis, Lyme disease, and Lupus;

FIG. 16 is a thermogram showing the effect of added bromocresol green on a plasma thermogram;

FIG. 17 includes Panel A, having a series a plots showing the differences between an average normal thermogram, and condition of interest thermograms, including Lupus (gray), Lyme disease (black), arthritis (thick black); and Panel B showing the difference between an average normal thermogram, and a thermogram generated using a normal plasma sample to which bromocresol green was added to a final concentration of 686 μM;

FIG. 18 includes Panel A, having plasma thermograms for a normal sample (gray), and samples to which bromocresol green was added to final concentrations of 30 μM (dashed), 148 μM (thick black), 290 μM (black) or 686 μM (circles); Panel C, having plots showing the differences in the thermograms of Panel A; Panel B, having thermograms for an HSA sample (gray), and an HSA sample to which bromocresol green was added to a final concentration of 459 μM (thick black); and Panel D, having a plot showing the differences in the thermograms of Panel B;

FIG. 19 includes a series of thermograms of samples from subjects with different stages of cervical cancer, where the top panel includes a black trace showing normal plasma, a gray trace showing a sample from a patient diagnosed with moderate cervical dysplasia (CIN II), and a dashed black trace showing a sample of plasma from a diagnosed cervical cancer patient, and where the bottom panel includes a single trace showing a thermogram for plasma from a Stage IVB cervical cancer patient;

FIG. 20 includes results from serum plasma electrophoresis of the samples used to obtain the data in FIG. 19, where the plasma protein fibrinogen is indicated by the asterisk, and where only subtle differences are evident between the panels and the most pronounced change is the relative increase in the globulin region of the electrophoresis pattern seen for the stage IVB sample (arrow);

FIG. 21 includes a series of thermograms generated using plasma samples obtained from different subjects, where the top panel includes thermograms generated using samples from four normal subjects, where the middle panel includes thermograms generated using samples from four subjects diagnosed with moderate cervical dysplasia (CIN II), where the bottom panel includes thermograms generated using samples from four subjects diagnosed with cervical cancer;

FIG. 22 includes thermograms for normal subjects, and subjects diagnosed with ovarian cancer, endometrial cancer, and uterine cancer;

FIG. 23 includes thermograms for subjects with melanoma;

FIG. 24 includes thermograms of plasma obtained prospectively from diabetic subjects exhibiting subsequent differences in future kidney function, and normal subjects exhibiting good kidney function (Panel A), and a quantile-quantile plot, prepared using the thermograms of Panel A (Panel B);

FIG. 25 includes thermograms of diabetic subjects with either minimal (CAD−) or severe (CAD+) coronary artery disease, and normal subjects;

FIG. 26 includes thermograms of subjects with amyotrophic lateral sclerosis (ALS), and normal subjects;

FIG. 27 includes an average thermogram generated using samples obtained from 100 normal subjects;

FIG. 28 includes a series of gender- and ethnic group-specific thermograms; and

FIG. 29 includes a series of quantile-quantile plots, prepared using the thermograms presented in FIG. 28, which illustrate the variation with gender and ethnicity.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the following terms are believed to be well understood by one of ordinary skill in the art, the following definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The presently-disclosed subject matter includes a method of diagnosing a condition of interest in a subject; a method of monitoring a condition of interest in a subject; a method for assessing the efficacy of a treatment program for a subject; a method of screening for compositions useful for treating a condition of interest; and a method of screening a composition for plasma protein interactions, including tendency of the composition to bind serum albumin.

As used herein, the term condition of interest refers to a variety of conditions. In some embodiments, the condition of interest can be cancer, including but not limited to cervical cancer, endometrial cancer, lung cancer, melanoma, multiple myeloma, ovarian cancer, and vulvar cancer. In some embodiments, the condition of interest can be an autoimmune disease, including but not limited to rheumatoid arthritis, multiple sclerosis, and systemic lupus. In some embodiments, the condition of interest can be caused by an infection, such as a bacterial or a viral infection; such conditions include but are not limited to Lyme disease, Dengue fever, and hepatitis. In some embodiments, the condition of interest can be another condition, including but not limited to amyotrophic lateral sclerosis (ALS), also known as Lou Gehrig's disease, anemia, cardiac disease, diabetes, renal disease, or plasma cell dyscrasias and related disorders. In some embodiments, the condition of interest can be a particular stage of a condition, for example, a particular stage of cervical cancer, such as moderate cervical dysplasia (CIN II), early stage cervical cancer, or stage IVB cervical cancer.

As used herein, the term subject refers to both human and animal subjects. Thus, veterinary therapeutic uses are provided in accordance with the presently-disclosed subject matter. As such, the presently-disclosed subject matter provides for the treatment of mammals such as humans, as well as those mammals of importance due to being endangered, such as Siberian tigers; of economic importance, such as animals raised on farms for consumption by humans or animals used for scientific research, such as rabbits, rats, and mice; and/or animals of social importance to humans, such as animals kept as pets or in zoos. Examples of such animals include but are not limited to: carnivores such as cats and dogs; swine, including pigs, hogs, and wild boars; rodents such as guinea pigs and hamsters; primates such as monkeys; arthropods including insects, arachnids and crustaceans; fish; mollusks; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The methods of the presently-disclosed subject matter make use of a unique calorimetric process for obtaining proteomic profiles of samples. Calorimetry provides a direct means for detecting what is perhaps the most fundamental property of chemical and biochemical reactions—heat changes. Biological calorimetry dates from the time of Lavoisier (1743-1794), who invented a calorimetric method for measuring the heats of metabolism of living animals. The presently-disclosed subject matter can make use of the high sensitivity of modern microcalorimeters, which can reliably measure heat changes of about 0.1 microcalories.

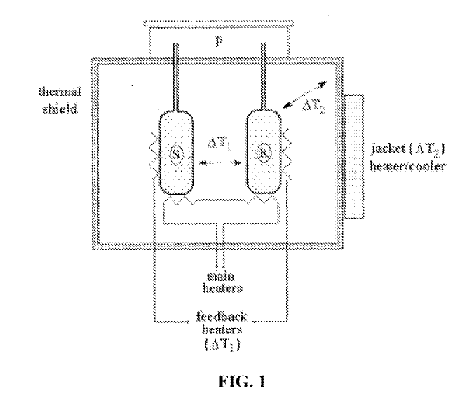

With reference to FIG. 1, an exemplary calorimeter that can be used in accordance with the presently-disclosed subject matter is a differential scanning calorimeter (DSC). In a typical DSC experiment, an aqueous solution of protein at a concentration of about 1 mg/mL or less is heated at a constant rate in a sample calorimeter cell (S) alongside an identical reference cell (R) that contains only the solvent (buffer). The electronics of the calorimeter are designed to maintain an exact thermal balance between the sample and references cells. Any chemical process in the sample cell that absorbs or releases heat results in a thermal imbalance with the reference cell, which is compensated for by a feedback heater attached to the calorimetric cells. The electrical power required to maintain the exact thermal balance of the cells is directly proportional to the apparent heat capacity of the solutions, and any change in the heat capacity is directly related to the energetics of the thermally-induced reactions that occur within the sample cell.

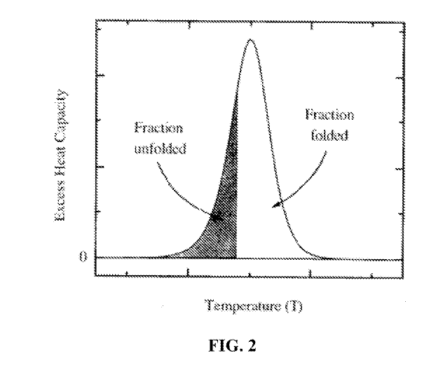

Differential scanning calorimetry (DSC) can be used for thermodynamic studies of protein denaturation. The thermodynamics of thermal-induced unfolding of proteins can be measured as directly as possible by DSC. With reference to FIG. 2, a thermogram can be obtained by DSC for a protein denaturation reaction, which expresses the excess heat capacity as a function of temperature. The area under such a thermogram is, unambiguously and directly, the enthalpy of the unfolding reaction. Integration of such a thermogram yields a transition curve (“melting curve”) from which the fractions of folded and unfolded forms can be calculated. The enthalpy obtained from the area of thermograms is independent of any model for the denaturation reaction that occurs in the sample cell. Such a calorimetric enthalpy provides a valuable alternative to enthalpy values obtained by use of the model-dependent van't Hoff equation (ΔH=−(δlnK/δT⁻¹) employing other methods, since no detailed reaction mechanism needs to be assumed. In other words, the calorimetric thermogram depends only on the initial and final states of the chemical system, and does not depend upon the manner in which the system passes from one state to the other.

Every protein has, under a given set of buffer conditions, a characteristic denaturation thermogram that is unique, and which provides a fundamental thermodynamic signature for that protein. Thermograms can be more complex than the simple two-state melting shown in FIG. 2. For more structurally complex proteins, individual structural domains within the tertiary structure can melt independently, leading to thermograms with correspondingly more complex shapes with multiple “peaks.”

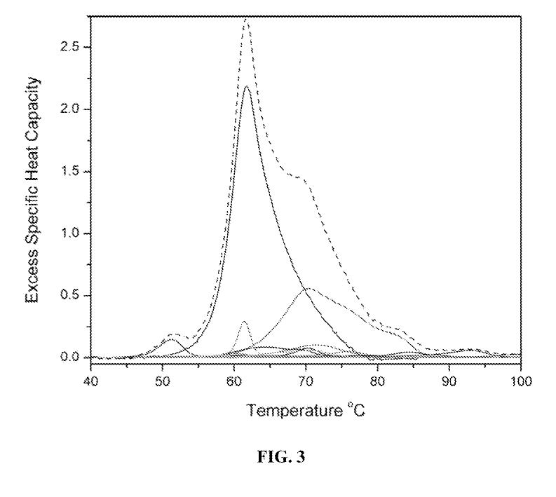

A primary DSC thermogram is an extensive property of a protein solution, and as such it is directly proportional to the mass of the protein in solution. If the weight concentration of the protein is doubled, for example, the calorimetric heat response will double. Similarly, in a solution of mixtures of proteins, the heat response will be proportional to the mass of each protein component in the mixture. Mixtures of proteins can be resolved with respect to the fundamental characteristic melting curves of their component proteins. Each protein in a noninteracting mixture will denature at its characteristic melting temperature (Tₘ) and with its characteristic melting enthalpy. The observed overall thermogram will be the weighted sum of all of the individual protein thermograms, weighted according to the mass of each component. For example, FIG. 3 contains thermograms for various individual proteins (solid lines), as well as a thermogram of the weighted sum of the group of individual proteins (dashed line).

Samples obtained from subjects, e.g., human plasma/serum samples, include mixtures of proteins. The presence of and the expression level of specific proteins in a mixture of proteins found in a sample can be referred to as the proteomic profile of the sample. The proteomic profile of a sample obtained from a subject having a condition differs from the proteomic profile of a normal subject, i.e., condition-free subject. As such, information about a subject of unknown status (having condition vs. normal/lacking condition) can be obtained by comparing a thermogram generated from a sample obtained from the subject to a thermogram generated from a sample associated with a known status.

Such thermograms have many advantages, for example: they are easily obtained on unlabeled, underivitized, unfractionated plasma/serum samples; they consume only modest amounts of sample; they are obtained relatively quickly; they are based on rigorous, fundamental physical properties of proteins within the sample; they are quantitative, and reflect the exact protein composition of the sample; the procedures for obtaining thermograms are amenable to automated, high-throughput screening; and they provide a new window for viewing plasma/serum composition, based on thermal stability rather than on molecular weight and charge as is the case for electrophoresis and mass spectrometry.

The methods of the presently-disclosed subject matter make use of signature thermograms and standard thermograms. As used herein, the term signature thermogram refers to a thermogram generated using a particular sample of interest. The sample of interest is often a sample obtained from a particular subject. In some embodiments, a method is provided for diagnosing or monitoring a condition of interest in a subject. In such embodiments, the signature thermogram can be a thermogram generated using a sample obtained from the subject being diagnosed or monitored. In some embodiments, a method of screening a composition for use in treating a condition of interest in a subject is provided. In such embodiments, the signature thermogram can be a thermogram generated using a sample obtained from the subject receiving the composition. In some embodiments, it can be desirable to obtain multiple signature thermograms. In such embodiments, the multiple signature thermograms are generated using samples of interest that are related in a particular manner. In such embodiments, samples of interest can be collected from the same subject (i.e., samples related in that they are obtained from the same subject) at different time points during the course of the treatment program.

As used herein, the term standard thermogram refers to a thermogram that is used as a reference to which a signature thermogram can be compared. A standard thermogram can be generated using a standard sample. A standard thermogram can be an average of multiple thermograms generated using multiple standard samples. For example, twenty standard samples can be obtained and a thermogram can be generated from each sample. The twenty generated thermograms could then be averaged to generate a standard thermogram.

In some embodiments, it can be desirable to provide a negative standard thermogram and/or a positive standard thermogram to which a signature thermogram can be compared. A negative standard thermogram is generated using a negative standard sample. For example, a negative standard thermogram can be generated using a sample known to be associated with an absence of a condition of interest, e.g., a sample obtained from a subject known not to have a condition of interest. A positive standard thermogram is generated using a positive standard sample. For example, a positive standard thermogram can be generated using a sample known to be associated with a presence of a condition of interest, e.g., a sample obtained from a subject known to have a condition of interest.

The standard thermogram can be generated using a standard sample obtained from a subject that is selected based on certain common characteristics relative to the subject. For example, if the subject from which a sample is obtained to generate a signature thermogram is a mouse, then the standard sample can be obtained from a mouse. For another example, if the subject from which a sample is obtained to generate a signature thermogram is a human, then the standard sample can be obtained from a human.

In some embodiments, it can be desirable to provide a group-specific standard thermogram to which a signature thermogram can be compared. A group-specific standard thermogram is a standard thermogram generated using a standard sample obtained from a member of the same identified group as the subject.

In some embodiments, when the subject is a member of a particular ethnic group or race, it is desirable to provide a group-specific standard thermogram generated using a sample obtained from a subject of the same ethnic group or race. For example, in some embodiments, when the subject is of Hispanic origin, it is desirable to provide an ethnic group-specific standard thermogram generated using a sample obtained from a subject of Hispanic origin. Other identified groups can include, for example, groups including members of African origin, of native American origin, of Asian origin, or of another ethnic group. In some embodiments, a group is identified by virtue of having negative standard thermograms that are good simulations of one another, i.e., where the standard thermograms of subjects who are substantially free of disease, sickness, or infection are good simulations of one another, a group can be identified to include these subjects.

In some embodiments, when the subject is a member of a particular sex, it can be desirable to provide a group-specific standard thermogram generated using a standard sample obtained from a subject of the same sex as the subject. For example, in some embodiments, when the subject is a female, it is desirable to provide a group-specific standard thermogram generated using a sample obtained from a female. In some embodiments, when the subject is a male, it is desirable to provide a group-specific standard thermogram generated using a sample obtained from a male.

A standard thermogram can be generated at a time point before, at a time point concurrent with or close to, or at a time point after the generation of a signature thermogram to which it will be compared. In some embodiments, it can be desirable to have a standard thermogram prepared to compare with various future-generated signature thermograms. In some embodiments, it can be desirable to provide a kit including one or more standard thermograms and instructions for generating signature thermograms for comparing with the one or more standard thermograms.

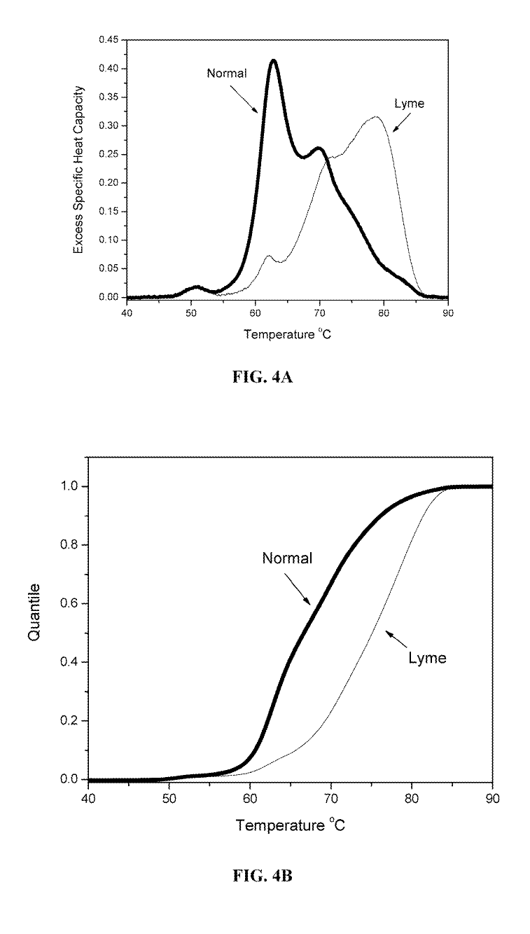

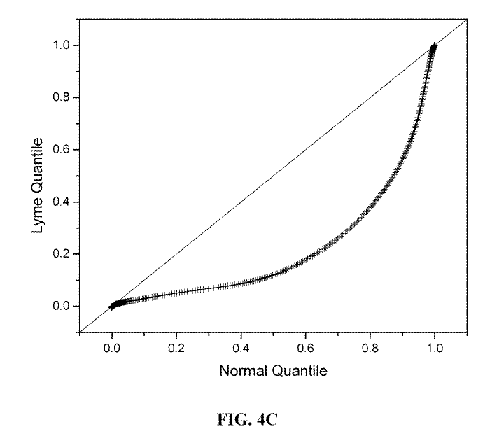

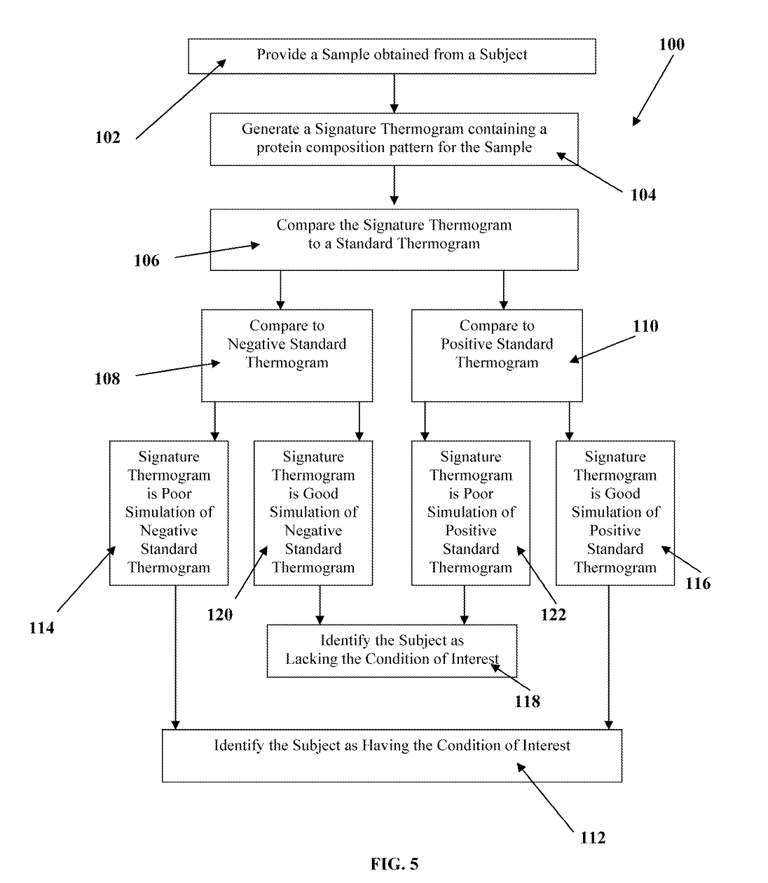

When comparing thermograms in accordance with methods of the presently-disclosed subject matter, they can be good simulations of one another or poor simulations of one another. When comparing thermograms, when a first thermogram is not a good simulation of a second thermogram, then it is a poor simulation of the second thermogram. A first thermogram is a good simulation of a second thermogram when it has substantial similarity to the second thermogram. In some embodiments, it is evident whether a first thermogram has substantial similarity to the second thermogram by inspection of the thermograms superimposed on one another, e.g., a signature thermogram superimposed on graphs of the standard(s). For example, FIG. 4A depicts a first normal thermogram (e.g., negative standard thermogram) and a second Lyme disease thermogram (e.g., signature thermogram) superimposed on one another. Upon inspection of the thermograms of FIG. 4A, it is evident that the first thermogram does not have substantial similarity to the second thermogram, i.e., poor simulation.

One of ordinary skill in the art can use his or her knowledge to make appropriate determinations of whether a substantial similarity can be found in particular situations.

In some embodiments, substantial similarity can be found when each of the peaks of the first thermogram occur at about the same temperatures as each of the peaks of the second thermogram. In some embodiments, substantial similarity can be found when the peaks of the first thermogram occur at temperatures within one standard deviation of the peaks of the second thermogram. In some embodiments, substantial similarity can be found when the peaks of the first thermogram occur at temperatures within two standard deviations of the peaks of the second thermogram.

In some embodiments, substantial similarity can be found when each of the peaks of the signature thermogram yield about the same heat capacity as the peaks of the standard thermogram. In some embodiments, substantial similarity can be found when the heat capacity of the peaks of the signature thermogram is within one standard deviation of the heat capacity of the peaks of the standard thermogram. In some embodiments, substantial similarity can be found when the heat capacity of the peaks of the signature thermogram is within two standard deviation of the heat capacity of the peaks of the standard thermogram.

In some embodiments, substantial similarity can be determined by application of published statistical procedures, for example, quantile-quantile plots (Lodder and Hieftje (1988)) can be used and/or a two-way Kolmogorov-Smirnov test can be used (Young (1977)). Briefly, for these tests, the thermogram must be converted to a quantile distribution. FIG. 4B depicts the quantile plots of the thermograms of FIG. 4A. Thermograms are converted to quantile distributions by the following steps: (1) the thermograms are baseline corrected and normalized thermograms are numerically integrated; (2) the integrated thermogram is normalized to 1.0; and (3) the resultant quantile plot thus consists of paired data points with temperature on the x axis and normalized quantile values on the y axis. To compare two thermograms, they must share common x values.

To construct a quantile-quantile plot, the quantile values derived from one thermogram is plotted against the quantile values derived from a second thermogram. FIG. 4C depicts a quantile-quantile plot generated using the quantile values of FIG. 4B, i.e., quantile for the first normal thermogram against the quantile for the second Lyme disease thermogram. If the two original thermograms are identical the paired data points will lie on a perfect straight line with a 45 degree angle from the origin. If the two original thermograms are not identical, points will deviate from the 45 degree straight line. As shown in FIG. 4C, the points for the Lyme disease quantile unambiguously and unacceptably deviate from the 45-degree straight line, indicating a poor simulation. In some embodiments, a first thermogram can be determined to be substantially similar to the second thermogram when the paired data points of the quantile-quantile plot lie on the 45 degree straight line, or have an acceptable deviation from the 45 degree straight line.

The same quantile values used to construct the quantile-quantile plot can be used to conduct a two-way Kolmogorov-Smirnov test, as implemented in standard statistical software packages and as is available online on service web sites (See, e.g., http://www.physics.csbsju.edu/stats/KS-test.html). The Kolmogorov-Smirnov test is designed to test the null hypothesis that two quantile distributions are not statistically different. The test returns a P-value for the confidence level with which the null hypothesis can be rejected. In this regard, in some embodiments, if the null hypothesis that the two quantile distributions are not statistically different (are good simulations) is rejected, it can be determined that the first thermogram is not substantially similar to the second thermogram. In some embodiments, the P-value is less than or equal to 0.5, 0.2, 0.1, 0.05, 0.02, 0.01, 0.005, 0.002, or 0.001.

By way of an example, when using the quantile values of FIG. 4B, a Kolmogorov-Smirnov test yields the results that the maximum between the cumulative distributions, D, is: 0.2028 with a corresponding P-value of less than 0.001, indicating that the null hypothesis that there is no difference between these quantile distribution can be rejected at the 99.999% confidence level, i.e., poor simulation.

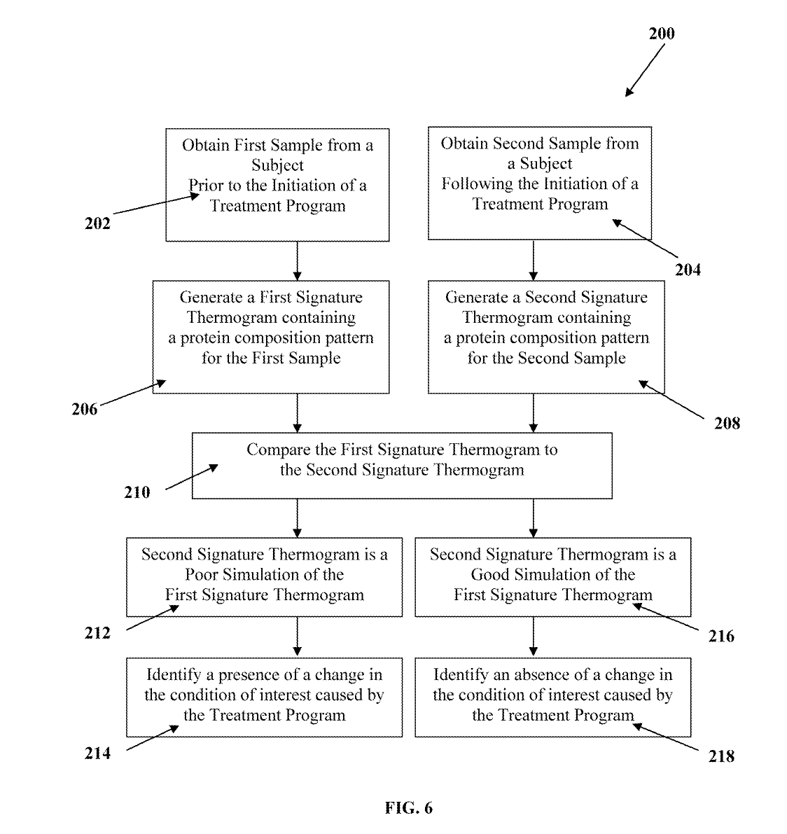

In some embodiments of the presently-disclosed subject matter, a method of diagnosing or monitoring a condition of interest in a subject is provided. With reference to FIG. 5, in some embodiments, a method of diagnosing or monitoring a condition of interest in a subject 100 includes, providing a sample obtained from the subject 102, generating a signature thermogram containing a protein composition pattern for the sample 104, comparing the signature thermogram to a standard thermogram 106, and identifying the subject as having the condition of interest 112 or identifying the subject as lacking the condition of interest 118.

As will be understood by those skilled in the art, the sample obtained from the subject 102 can be any appropriate biological sample, such as a body fluid. Appropriate body fluids include, ascites fluid, blood, cerebral spinal fluid, serum, peritoneal fluid, plasma, saliva, senovial fluid, ocular fluid, urine, and the like. As will be understood by those skilled in the art, in some cases it can be desirable to select the type of sample being collected based on the selected condition of interest. For example, in some embodiments when the condition of interest is ALS, it can be desirable to obtain a cerebral spinal fluid sample.

In some embodiments, an obtained sample can be prepared in the following manner. A blood sample is drawn from the subject and plasma or serum is isolated from the blood using known methods. A small volume of about 100 μL of plasma or serum is dialyzed at about 4° C. against a standard buffer (e.g., 10 mM potassium phosphate, 150 mM NaCl, 0.38% (w/v) sodium citrate, pH 7.5 for plasma; 10 mM potassium phosphate, 150 mM NaCl, pH 7.5 for serum). Dialyzed plasma or serum is filtered to remove particulates and then diluted about 25-fold into the standard buffer.

With continued reference to FIG. 5, the prepared sample can be used to generate a signature thermogram containing a protein composition pattern for the sample 104. The sample is run on a differential scanning calorimeter (DSC) to obtain a thermogram for the sample. A differential scanning calorimeter (DSC) can be obtained from MicroCal, LLC (Northampton, Mass.), for example, the MicroCal, LLC VP Capillary Differential Scanning Calorimeter can be used. Any differential scanning calorimeter (DSC) with the requisite sensitivity, temperature range, scanning rate, and baseline stability could be used in accordance with the methods of the presently-disclosed subject matter. Examples of other instruments that would be suitable include: Calorimetric Sciences Corporation's N-DSCII Differential Scanning Calorimeter, TA Instruments Incorporated's Q2000 Differential Scanning Calorimeter, and Perkin Elmer Corporation's Diamond DSC Differential Scanning Calorimeter. Newly designed instruments that might become available with the requisite sensitivity, temperature range, scanning rate, and baseline stability could also be used to practice the methods of the presently-disclosed subject matter. For example, Energetic Genomics Corporation's 96-well differential scanning calorimeter that is under development, and for which a prototype instrument is available to the inventors, could be used to practice the methods of the presently-disclosed subject matter. Standard software and protocols can be used to obtain a thermogram for the sample with the selected DSC.

Using the MicroCal, LLC DSC as an example, a sample volume of approximately 0.4 mL is needed for liquid-handling for proper filling of the sample cell, although the effective cell volume is only approximately 0.133 mL. Each DSC run takes about 1-2 hours to complete. Total protein concentrations of the diluted sample can be determined by standard colorimetric, spectrophotometric, or refractometric methods. These concentrations can be used to normalize experimental thermograms to a g/L protein concentration scale. This normalized thermogram shows the “Excess Specific Heat Capacity” as a function of temperature for a plasma/serum sample (See, e.g., the dashed line thermogram of FIG. 3). Such a thermogram provides a specific signature for a particular sample that provides a snapshot of the protein composition of the sample.

With continued reference to FIG. 5, once the signature thermogram is generated, it can be compared to a standard thermogram 106. To minimize uncontrolled variables, the sample used to generate the signature thermogram should be prepared in the same manner as the sample used to generate the standard thermogram. Similarly, the calorimeter, software, and protocols used to generate the signature thermogram should be substantially the same as those used to generate the standard thermogram. The standard thermogram can be a negative standard thermogram 108, in that it is associated with an absence of the condition of interest. The negative standard thermogram can be generated using a sample obtained from a subject who is “normal,” i.e., condition-free. In some cases the sample can have been obtained from the subject at a time when that subject was known to be condition-free. The standard thermogram can also be a positive standard thermogram 110, in that it is associated with a presence of the condition of interest. The positive standard thermogram can be generated using a sample obtained from a subject who has the condition of interest. In some cases the sample can have been obtained from the subject at a time when that subject was known have the condition. In some embodiments, the signature thermogram can be compared to both a negative standard thermogram and a positive standard thermogram.

In some embodiments, the subject can be identified as having the condition of interest 112 when the signature thermogram is compared to a negative standard thermogram, and is found to be a poor simulation of the negative standard thermogram 114. In some embodiments, the subject can be identified as having the condition of interest 112 when the signature thermogram is compared to a positive standard thermogram, and is found to be a good simulation of the positive standard thermogram 116. In some embodiments, the subject can be identified as having the condition of interest 112 when the signature thermogram is compared to a positive standard thermogram and a negative standard thermogram, and is found to be a good simulation of the positive standard thermogram 116 and a poor simulation of the negative standard thermogram 114.

In some embodiments, the subject can be identified as lacking the condition of interest 118 when the signature thermogram is compared to a negative standard thermogram and is found to be a good simulation of the negative standard thermogram 120. In some embodiments, the subject can also be identified as lacking the condition of interest 118 when the signature thermogram is compared to a positive standard thermogram and is found to be a poor simulation of the positive standard thermogram 122. In some embodiments, the subject can also be identified as lacking the condition of interest 118 when the signature thermogram is compared to a negative standard thermogram and a positive standard thermogram, and is found to be a good simulation of the negative standard thermogram 120 and a poor simulation of the positive standard thermogram 122.

In some embodiments, the subject can be identified as having a condition, albeit unidentified for the time being, when the signature thermogram is found to be a poor simulation of the negative standard thermogram. Upon such a finding, the signature thermogram can then be compared to positive standard thermograms associated with conditions of interest in order to make a diagnosis.

In some embodiments, the signature thermogram can be compared to multiple positive standard thermograms, e.g., a database including multiple positive standard thermograms, each positive standard thermogram being associated with a particular condition of interest. The positive standard thermogram that most resembles the signature thermogram can be selected. The subject can be identified as having the condition associated with the positive standard thermogram that most resembles the signature thermogram. In some embodiments, the method can be useful to distinguish between two conditions having initial symptoms that are difficult to distinguish; for example, in some embodiments, the method can be used to distinguish multiple sclerosis and ALS in a subject.

As will be understood by those of ordinary skill in the art, it can sometimes be desirable to obtain multiple samples from the subject at various time points, in order to monitor the condition of interest. For example, in some embodiments, a second sample can be obtained from the subject at a time point after the first sample is obtained. A second signature thermogram containing a protein composition pattern for the second sample can be generated. The first signature thermogram can be compared to the second signature thermogram. The condition of interest can be identified as changed when the second signature thermogram is a poor simulation of the first signature thermogram. The condition of interest can be identified as unchanged when the second signature thermogram is a good simulation of the first signature thermogram.

In some embodiments, the second signature thermogram can also be compared to a negative standard thermogram. If the second signature thermogram is a good simulation of the negative standard thermogram, for a subject that had previously been identified as having a particular condition, the subject can be identified as having improved to the point of lacking the condition. In some embodiments, the second signature thermogram can also be compared to various positive standard thermograms associated with different stages of a particular condition. In this regard, it can be determined whether the condition is progressing, i.e., becoming more severe, or regressing, i.e., improving.

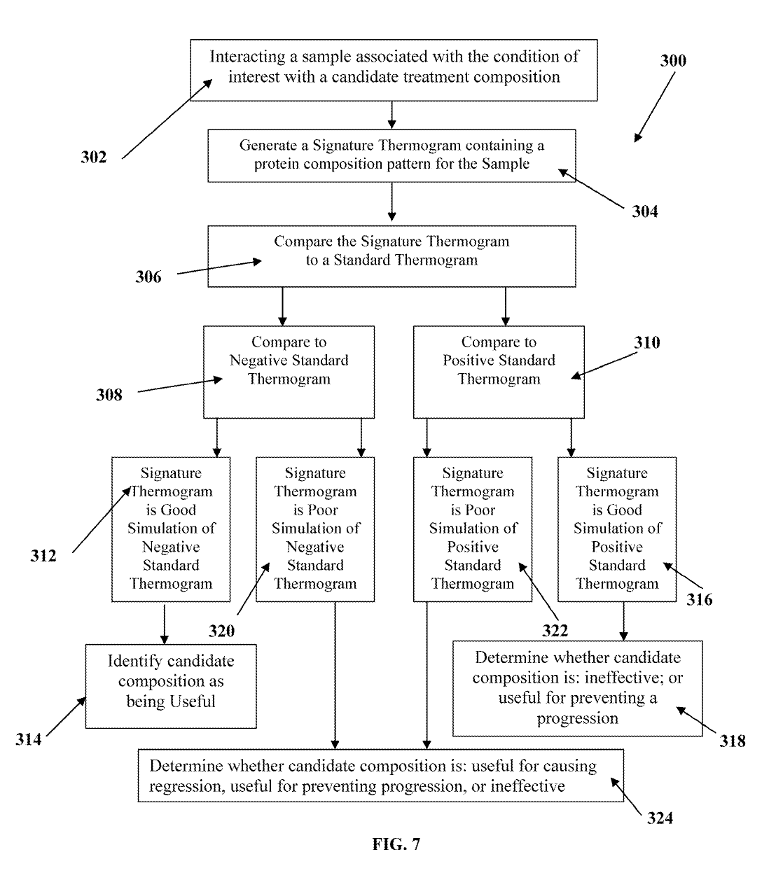

With reference now to FIG. 6, the presently-disclosed subject matter includes a method of assessing efficacy of a treatment program for a subject 200. As used herein, a treatment program includes a plan for treating a subject or providing treatment to a subject. As used herein, the terms treatment or treating relate to any treatment of a condition of interest, including but not limited to prophylactic treatment and therapeutic treatment. As such, the terms treatment or treating include, but are not limited to: preventing the development of a condition of interest; inhibiting the progression of a condition of interest; arresting or preventing the development of a condition of interest; reducing the severity of a condition of interest; ameliorating or relieving symptoms associated with a condition of interest; and causing a regression of the condition of interest or one or more of the symptoms associated with the condition of interest. As will be understood by those of ordinary skill in the art, a treatment program can differ depending on the condition of interest and the subject being treated. A treating physician can select a particular treatment program based on the condition of interest, and the particular subject being treated. Depending on the situation, a treatment program could include, for example, administering a treatment composition or a series of treatment compositions, administering a radiation treatment, prescribing an altered diet, prescribing a particular exercise regimen, prescribing low activity or rest, a combination thereof, etc.

In some embodiments, a method of assessing efficacy of a treatment program for a subject 200 includes the following: obtaining a first sample from the subject prior to the initiation of the treatment program 202, obtaining a second sample from the subject following the initiation of the treatment program 204, generating a first signature thermogram using the first sample 206, generating a second signature thermogram using the second sample 208, comparing the first signature thermogram to the second signature thermogram 210, and identifying the presence or absence of a change in the condition of interest 214, 218.

The first sample is obtained from the subject before initiation of the treatment program 202 and is used to generate a first signature thermogram containing a protein composition pattern 206. In some embodiments, the subject has a condition of interest when the first sample is collected. In some embodiments, the subject does not have a condition of interest, but there is otherwise a reason for receiving a treatment program, as will be understood by those of ordinary skill in the art. For example, a subject lacking a condition of interest, but having a risk for obtaining the condition of interest could receive a treatment program, the efficacy of which can be assessed using the method of the presently-disclosed subject matter.

The second sample is obtained from the subject following the initiation of the treatment program 204 and is used to generate a second signature thermogram containing a protein composition pattern 208 associated with the treatment program of the subject. For example, the treatment program could include administration of a treatment composition and the second sample could be obtained after the subject has been receiving the treatment composition for a day, week, month, or other time period of interest. For another example, the treatment program could include providing radiation treatment and the second sample could be obtained after the subject has been receiving the radiation treatment for a specific period of time. In any event, the second sample is obtained at a time point of interest after the treatment program has been initiated. Additional samples can be obtained at different time points of interest to generate a time course describing the effect of the treatment program on the subject.

The signature thermograms are generated 206, 208 by running the samples on a differential scanning calorimeter (DSC) to obtain thermogram for the samples. Once the signature thermograms are generated, they are compared to one another 210. To minimize uncontrolled variables, the sample used to generate the first signature thermogram should be prepared in the same manner and be of the same type as the sample used to generate the second signature thermogram. Similarly, the calorimeter, software, and protocols used to generate the signature thermogram should be substantially the same as those used to generate the standard thermogram.

When the signature thermograms are compared and the second signature thermogram is found to be a good simulation of the first signature thermogram 216, then the treatment program can be identified as having not changed the condition of the subject 218, i.e., absence of a change.

When the signature thermograms are compared and the second signature thermogram is found to be a poor simulation of the first signature thermogram 212, then the treatment program can be identified as having changed the condition of the subject 214, i.e., presence of a change.

As will be understood by those of ordinary skill in the art, depending on the goal of the treatment program, an absence or a presence of a change can be indicative of an effective or an ineffective treatment program. As such, the determination of whether the presence or absence of a change is indicative of an effective treatment program will differ depending on the goal of the treatment program.

In some embodiments, when there is an absence of a change, the treatment program can be identified as an effective treatment program. In some embodiments, when there is an absence of a change, the treatment program can be identified as an ineffective treatment program. For example, if a prophylactic treatment program is administered to a subject lacking a condition of interest, with a goal of preventing an onset of the condition of interest, an absence of a change in the condition of the subject can be indicative of an effective (successful) treatment program. For another example, if a therapeutic treatment program is administered to a subject having a condition of interest, an absence of a change in the condition of the subject can be indicative of an effective treatment program if the goal is to prevent progression of the condition, or an ineffective treatment program if the goal is to cause a regression of the condition.

In some embodiments, when there is a presence of a change, the treatment program can be identified as an effective treatment program. In some embodiments, when there is a presence of a change, the treatment program can be identified as an ineffective treatment program. For example, in some embodiments, a prophylactic treatment program is administered to a subject who initially lacked a condition of interest; in such embodiments, a change in the condition can be indicative of an ineffective treatment program.

In some embodiments, it is apparent by inspecting the thermograms whether a change is indicative of an effective or an ineffective treatment program, e.g., change indicative of a regression of a condition, or a progression of a condition, as will be understood by those of ordinary skill in the art. In some embodiments, it can be desirable to additionally compare the signature thermogram to one or more standard thermograms. For example, in some embodiments a treatment program is administered to a subject who initially had a condition of interest; in such embodiments, a change in the condition can be indicative of either a regression or a progression of the condition. In such cases, as will be understood by those of ordinary skill in the art, it can be useful to additionally compare the second signature thermogram to one or more standard thermograms. For example, if the second signature thermogram is a good simulation of a negative standard thermogram, then the change can be indicative of a regression. In some embodiments, it can be useful to compare the second signature thermogram to a series of positive standard thermograms, each associated with a particular stage of the condition of interest. Such comparisons can also provide information about whether a change in the condition is indicative of a progression or a regression of the condition.

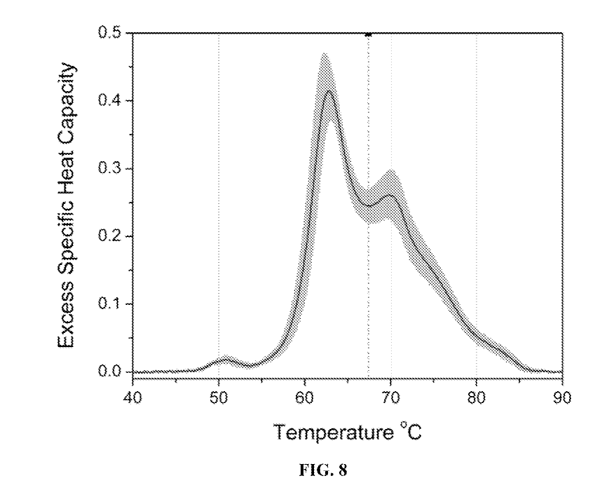

With reference now to FIG. 7, the presently-disclosed subject matter includes a method of screening for a composition useful for treating a condition of interest 300. In some embodiments, the method includes: interacting a sample associated with the condition of interest with a candidate treatment composition 302, generating a signature thermogram containing a protein composition pattern for the sample 304, comparing the signature thermogram to a standard thermogram 306, and determining the utility of the candidate treatment composition 314, 318, 324.

With regard to the step of interacting a sample associated with the condition of interest with a candidate treatment composition 302, in some embodiment, the candidate treatment composition can be administered to an infected subject 302. The subject can be any appropriate test subject, for example, a mouse, a rat, a rabbit, or another appropriate test subject. In some embodiments, the candidate treatment composition can be administered to a subject that is a model for a condition of interest, e.g., mouse model for a particular condition. The candidate composition can be administered by any appropriate method, depending on the characteristics of the composition being screened. A sample, e.g., body fluid sample, can then be obtained from the test subject for use in generating the signature thermogram. In some embodiments, the step of interacting a sample associated with the condition of interest with a candidate treatment composition includes administering the candidate treatment composition to cells in culture, which cells have been infected with or are otherwise associated with the condition of interest. A sample can then be extracted from the cells for use in generating the signature thermogram. The signature thermogram containing a protein composition pattern for the sample can be generated 304 using a differential scanning calorimeter (DSC).

With continued reference to FIG. 7, once the signature thermogram is generated, it can be compared to a standard thermogram 306. To minimize uncontrolled variables, the sample used to generate the signature thermogram should be prepared in the same manner and obtained from the same species as the sample used to generate the standard thermogram. Similarly, the calorimeter, software, and protocols used to generate the signature thermogram should be substantially the same as those used to generate the standard thermogram.

The standard thermogram can be a negative standard thermogram 308, in that it is associated with an absence of the condition of interest. The negative standard thermogram can be generated using a sample associated with an absence of the condition of interest, e.g., a sample obtained from a subject who is “normal,” or condition-free. In some embodiments, the negative standard sample can be obtained from a subject administered the candidate treatment composition, in which case it is obtained prior to the infection of the subject and prior to administration of the candidate treatment composition.

The standard thermogram can also be a positive standard thermogram 310, in that it is associated with a presence of the condition of interest. In some embodiments, the positive standard thermogram can be generated using a sample obtained from a subject who has the condition of interest. In some embodiments, the positive standard sample can be obtained from the subject administered the candidate treatment composition, in which case it is obtained after the subject is infected and prior to administration of the candidate treatment composition.

In some embodiments, the signature thermogram is a good simulation of the negative standard thermogram 312 associated with an absence of the condition of interest, and the candidate treatment composition can be identified as being useful 314.

In some embodiments, the signature thermogram is a good simulation of the positive standard thermogram 316 associated with a presence of the condition of interest. It can then be determined whether the candidate treatment composition is either useful for preventing a progression of the condition, or is ineffective if the goal is to cause a regression of the condition 318.

In some embodiments, the signature thermogram is a poor simulation of the negative standard thermogram 320 and/or a poor simulation of the positive standard thermogram 322. It can then be determined whether the candidate treatment composition is either useful for causing a regression of the condition, useful for preventing a progression of the condition, or is ineffective, i.e., not treatment affected, or causes a progression of the condition 324.

In order to make the determination of whether the candidate treatment composition is useful for causing a regression of the condition, useful for preventing a progression of the condition, or is ineffective, it can be desirable to obtain a series of samples collected over time, for use in generating a series of signature thermograms. The series of signature thermograms can be compared to identify any changes. In some embodiments, it is apparent by inspecting the series of signature thermograms whether a change is indicative of an effective or an ineffective treatment program. For example, if the series of signature thermograms display a trend towards a good simulation of the negative standard thermogram, then it can be determined that the candidate treatment composition causes a regression of the condition. For another example, if the series of signature thermograms display no change, then it can be determined that the candidate treatment composition prevents a progression of the condition. For another example, if the series of signature thermograms display a trend towards a good simulation of the positive standard thermogram, then it can be determined that the candidate treatment composition neither causes a regression of the condition nor prevents a progression of the condition, i.e., ineffective.

In some embodiments, it can be desirable to additionally compare the signature thermogram to one or more standard thermograms. In some embodiments, the series of signature thermograms can be compared to one or more positive standard thermograms associated with different stages of a condition of interest. For example, if the condition of interest is cervical cancer, standard thermograms associated with moderate cervical dysplasia (CIN II), early stage cervical cancer, and stage IVB cervical cancer could be provided. The series of signature thermograms could be used to determine whether the candidate treatment composition affects a regression of the cervical cancer from stage IVB cervical cancer, to early stage cervical cancer, to moderate cervical dysplasia; a progression from moderate cervical dysplasia, to early stage cervical cancer, to stage IVB cervical cancer; or no change.

In some embodiments, the candidate treatment composition can be administered to a test subject before the test subject has been infected with the condition of interest. The subject can then be infected, samples obtained, and thermograms generated. The thermograms can be compared to determine the ability of the candidate treatment composition to prevent or inhibit an onset or progression of a condition of interest.

The presently-disclosed subject matter further includes a method of screening a composition, e.g. candidate drug or treatment, for protein interactions, to identify and/or monitor the capacity of the composition to interact with protein. In some embodiments, the method includes: interacting the composition with a sample; generating a signature thermogram containing a protein composition pattern for the first sample; comparing the signature thermogram to a thermogram containing a protein composition pattern associated with an absence of protein interactions; identifying the candidate composition as lacking substantial plasma protein interactions when the first signature thermogram is a good simulation of the thermogram containing a protein composition pattern associated with an absence of protein interactions.

In some embodiments, the thermogram containing a protein composition pattern associated with an absence of protein interactions can be a negative standard thermogram. In some embodiments, the thermogram containing a protein composition pattern associated with an absence of protein interactions can be a second signature thermogram generated using a second sample not interacted with the composition.

In some embodiments, the sample is a plasma sample or a serum sample. In such embodiments, the method can be used to identify and/or monitor capacity of composition, e.g., candidate drug, to bind serum albumin and/or other serum or plasma protein interactions.

During drug development and efficacy studies, it can be desirable to identify and monitor interactions between a compound of interest (e.g., drug candidate) and components of plasma. For example, it will be appreciated by those of ordinary skill in the art that it can be desirable to identify and/or monitor a compound of interest for binding to serum albumin.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

Reproducible Thermogram for normal plasma. FIG. 8 shows an average thermogram obtained from plasma samples from 15 normal subjects. The thermogram displays multiple peaks and shoulders, yet is surprisingly simple, given the complexity of the plasma proteome. The average thermogram is shown as the black trace, and the standard deviation from the mean appears as the shaded region of FIG. 8. The standard deviation of the data is low, and is comparable to the range in values observed in normal subjects for the concentrations of individual plasma proteins (Craig (2004)). Human serum albumin, for example, has a normal reference range of approximately 35 to 55 g/L, dependent on age and gender (Craig (2004)). This analysis indicates that thermograms from normal subjects are highly reproducible. As noted below, the thermograms for samples associated with various conditions of interest all deviate beyond the range of normal values of the thermogram of FIG. 8, and their patterns must be considered to be significantly different from normal.

The average normal thermogram in FIG. 8 shows clear peaks at 50.8, 62.8 and 69.8° C. The area under the thermogram is 5.02±0.23 cal g⁻¹, and defines the specific enthalpy for the denaturation of normal plasma over the range 45-90° C. The first moment of the thermogram with respect to the temperature axis is 67.4±0.8° C. The sample size used in these studies is appropriate for exploratory preclinical studies, and, indeed, is on par with the numbers expected for a Phase I clinical trial (Motulsky (1995)).

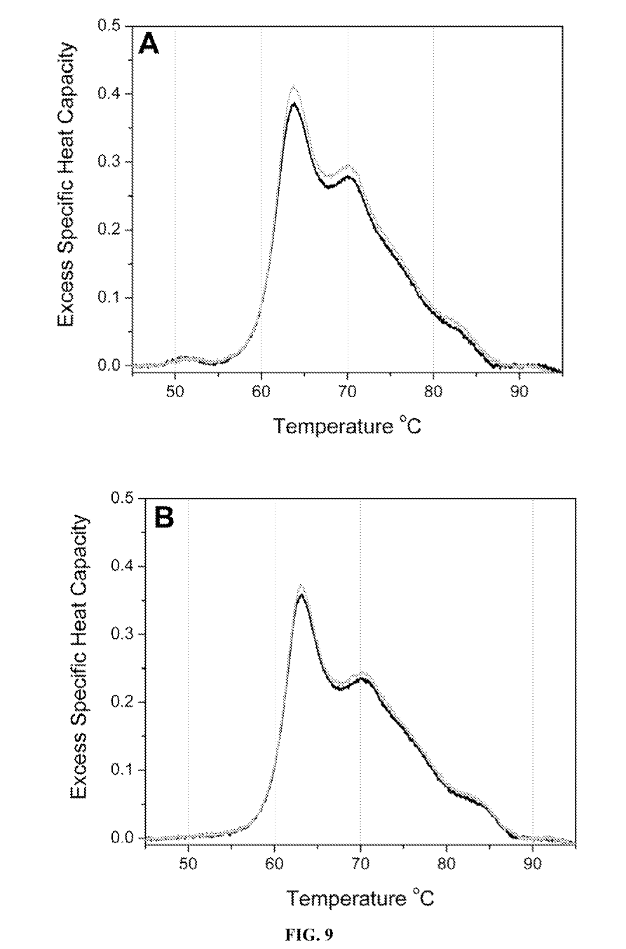

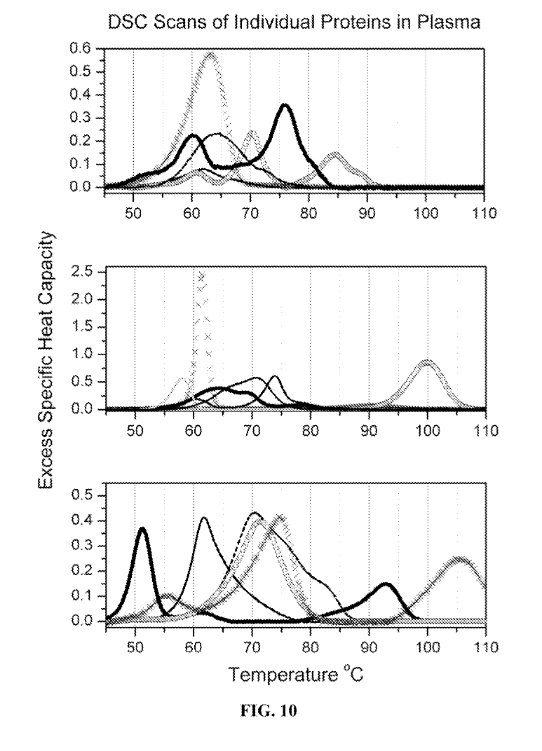

In order to establish that frozen samples can be thawed and used in accordance with the methods of the presently-disclosed subject matter, thermograms were generated for freshly prepared samples, and compared to thermograms generated using samples that were thawed after being frozen. With reference to FIG. 9 (Panel A), the solid gray line shows the thermogram of the thawed plasma sample (after being frozen at −20° C.), and the solid black line shows the thermogram for the freshly-prepared plasma sample. The differences are well within the standard deviation obtained for the average normal thermogram. With reference to FIG. 9 (Panel B), solid gray line shows the thermogram of the thawed serum sample (after being frozen at −20° C.), and the solid black line shows the thermogram for the freshly-prepared serum sample. Again, the differences are well within the standard deviation obtained for the average normal thermogram. Note the small transition ˜51° C. in the plasma representing the melting of one domain of fibrinogen; this peak is absent in serum.

Normal plasma thermogram is the weighted sum of the denaturation of individual plasma proteins. Applicants hypothesized that the thermogram seen in FIG. 8 arises from the denaturation of the individual proteins within plasma, and represents the sum of individual protein denaturation reactions weighted according to their concentrations within plasma.

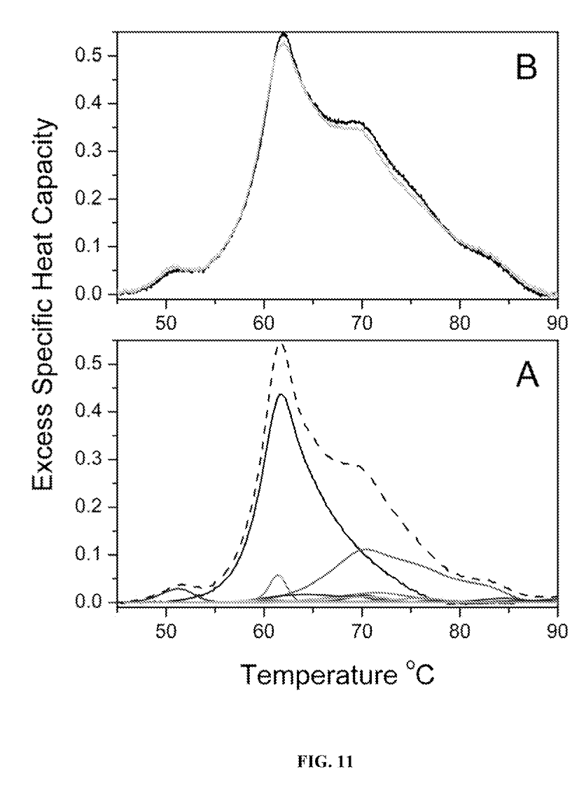

This hypothesis was tested in two ways. With reference to FIG. 10, individual thermograms for the denaturation of the sixteen (16) most abundant plasma proteins were determined. FIG. 10 includes a series of thermograms of individual purified plasma proteins. The top panel shows superimposed thermograms for α₁-antitrypsin (black), transferrin (circles), α₁-acid glycoprotein (dashed), complement C3 (thick black), and c-reactive protein (crosses). The middle panel shows thermograms for haptoglobin (crosses), prealbumin (circles), α₂-macroglobulin (thick black), complement C4 (black), α₁-antichymotrypsin (gray), and IgM (dashed). The bottom panel shows thermograms for albumin (black), IgG (dashed), fibrinogen (thick black), IgA (circles), and ceruloplasmin (crosses). These thermograms display a range of denaturation temperatures, and differences in the complexities of their denaturation reactions. Many of these thermograms show multiple peaks, indicative of complex denaturation reactions, while other thermograms are consistent with simple two-state melting behavior.

FIG. 11 (Panel A) shows the calculated plasma thermogram obtained by simple summation of the individual thermograms for the 16 most abundant plasma proteins after weighting their contribution according to their known average concentrations in normal plasma (Craig (2004)). Multicomponent analysis was used. A tacit assumption in this exercise is that there are no interactions among these proteins that might alter their thermal denaturation. The resultant shape of the calculated thermogram mimics that of the experimental one seen in FIG. 8, in support of the Applicants' hypothesis.

Referring now to FIG. 11 (Panel B), as a second test, mixtures of pure individual plasma proteins were prepared, and their thermograms determined by DSC. A mixture containing the 16 most abundant plasma proteins at their average concentrations found in normal plasma yields a thermogram whose shape mimics that of actual plasma (black curve of FIG. 11 (Panel B)). A mixture with only the four (4) major components (HSA, IgG, fibrinogen and transferrin) yields a thermogram that closely matches the observed normal, but which lacks subtle features (gray curve of FIG. 11 (Panel B)).

The data presented in FIG. 11 show that the normal thermogram is dominated by contributions from those four proteins. The small peak at 50.8° C. can be unambiguously assigned to a transition in fibrinogen. The major peak at 62.8° C. primarily reflects the denaturation of unligated HSA, with a contribution from haptoglobin. The peak a 69.8° C. and the shoulders at higher temperature arise primarily from IgG.

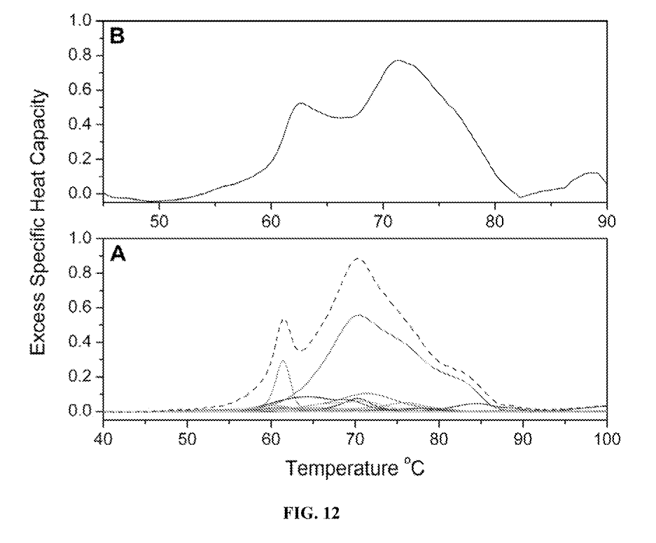

Thermograms of HSA-depleted serum. FIG. 12 shows the results from experiments in which albumin was removed from serum by affinity chromatography. (Serum differs from plasma primarily by the absence of fibrinogen, which is removed when plasma is allowed to clot.) FIG. 12 (Panel A) shows an expected thermogram (dashed line) obtained by calculating the weighted sum of the most abundant proteins (solid lines), minus HSA and fibrinogen. FIG. 12 (Panel B) shows the observed experimental thermogram for albumin-depleted serum. The agreement between the shape of the calculated and observed thermograms is excellent. Apart from confirming the major contribution by HSA to the peak at 62.8° C. in plasma thermograms, these data show that the contributions of other plasma proteins to thermograms can be amplified for more detailed study.

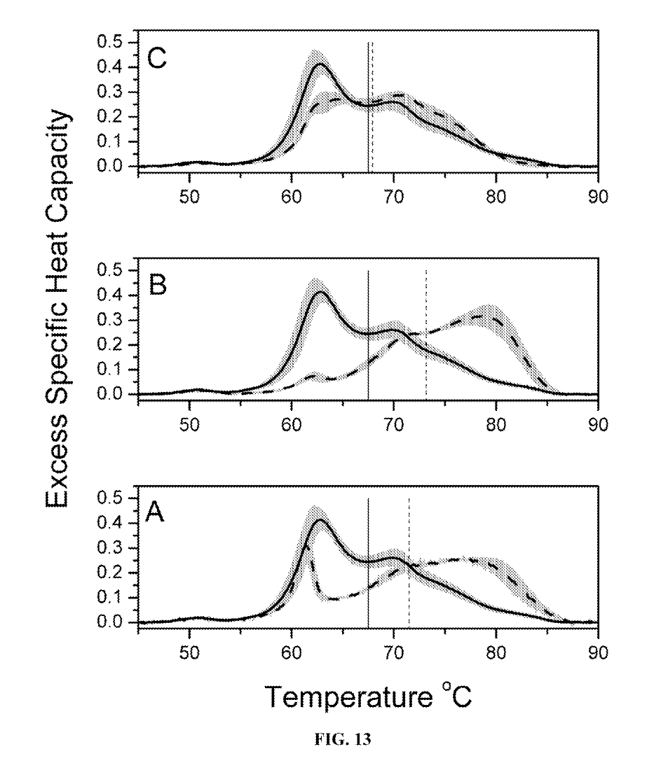

Distinctive thermograms for samples associated with a condition of interest. Plasma samples for subjects suffering from various conditions were obtained from BBI Diagnostics (West Bridgewater, Mass.). For comparison, plasma samples from 15 normal subjects were studied. Thermograms were obtained and compared as described herein, and the results are shown in FIG. 13. Shading indicates the standard deviation of the excess specific heat capacity at each temperature. The thermograms of diseased plasma (dashed lines) are distinctly different from thermograms obtained for plasma from normal subjects (solid lines). In addition, the thermograms for the diseased plasmas differ from one another, each showing distinctive patterns. FIG. 13 specifically compares average thermograms for subjects with three different conditions (rheumatoid arthritis, Lyme disease, systemic lupus) with the average normal thermogram. As noted, each disease appears to display a signature thermogram that differs from other diseases. In all cases, the 62.8° C. peak associated with HSA is greatly diminished, and the thermograms are shifted to higher temperatures. The solid vertical line is the first moment of the normal thermogram and the dashed vertical line is the first moment of the diseased thermogram.

FIG. 13 (Panel A) shows the thermogram for lupus. The first moment shifts from the normal value of 67.5 to 71.5° C. A sharp peak near 61° C. is evident that would be consistent with an elevation in haptoglobin concentration.

The thermogram for Lyme disease (FIG. 13 (Panel B)) is distinct from that seen for systemic lupus. The first moment at 73.15° C. is higher still, and the shape of the thermogram clearly differs from both normal and lupus thermograms.

FIG. 13 (Panel C) shows yet another distinctive thermogram for subjects suffering from rheumatoid arthritis. That thermogram is characterized by a first moment of 67.9° C., only slightly higher than normal, but with distinct changes in the shape relative to normal that are well beyond the standard deviations in the two thermograms. These collective results establish that embodiments of the methods of the presently-disclosed subject matter are useful and efficacious as clinical diagnostic tools. Thermograms can at a glance distinguish diseased states from normal, and have the potential for providing signatures for any specific condition of interest. The samples sizes used in these studies conform to the accepted standards for exploratory preclinical studies (Motulsky (1995)).

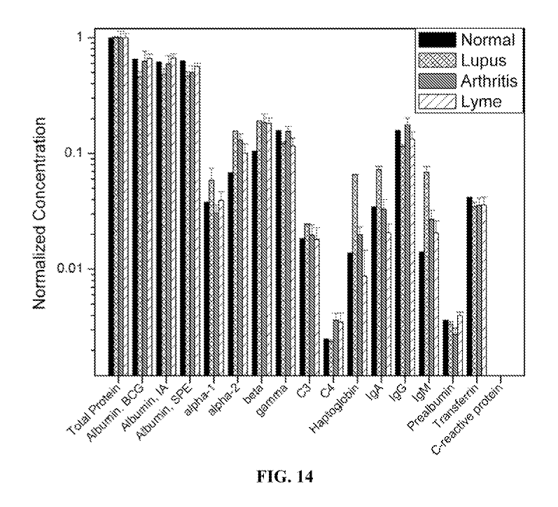

Origin of the altered thermograms. What causes the dramatic alterations in thermograms seen in FIG. 13? One possibility is that the concentrations of the major proteins in plasma are changed. This possibility was tested by experiments, and it was found that such is not the case. FIG. 14 shows the concentrations of the major plasma proteins for the same samples shown in FIG. 13. The data show that the protein composition of plasma from diseased subjects is in most cases indistinguishable from normal concentration values. Plasma from lupus patients represents a slight exception, with samples showing elevated concentrations of haptoglobin, IgA and IgM. Notably, albumin concentrations are normal for all of the diseased states, even though the thermogram peak at 62.8° C. that is characteristic of albumin is absent or greatly diminished in diseased samples (FIG. 13).

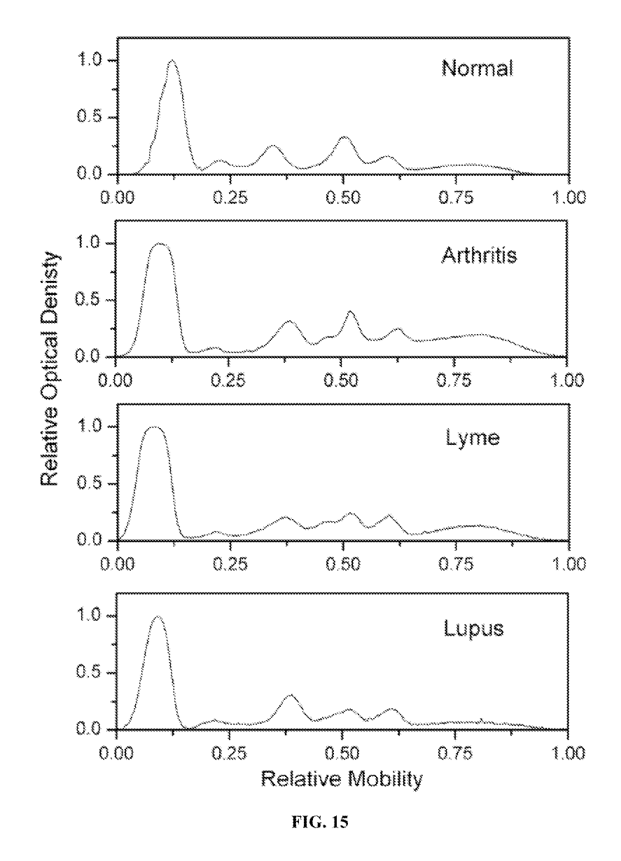

FIG. 15 shows protein electrophoresis patterns for normal plasma and the diseased states. Only subtle variations can be seen when comparing these traces, in contrast to the dramatic shifts in thermograms seen in FIG. 13. These data reveal a distinct advantage of the methods described herein. While whatever is present in plasma in the diseased state that differentiates samples from normal does not seem to drastically alter the concentrations or the sizes and charges of the plasma proteins (as revealed by electrophoresis), it does exert dramatic effects on the thermal properties of the proteins.

The most likely explanation for the shifts in the thermograms in FIG. 13 is that it results from binding interactions that involve the most abundant plasma proteins, particularly albumin. This view is consistent with the “interactome” hypothesis, that suggests that peptide and protein biomarkers specific for a particular disease are not free in plasma, but rather are bound to albumin or the immunoglobins. Such binding would result in thermal stabilization of the protein to which the biomarkers are bound, and a drastic alteration of the plasma thermogram with respect to normal. That is exactly what is seen in FIG. 13.

In order to test the hypothesis that shifted thermograms result from interactions, the following study was performed. Bromocresol green is a small organic molecule that binds to Site I of human serum albumin (HSA) with a binding constant of 7×10⁵ M⁻¹ (Peters (1996)). The consequences of such binding on plasma thermograms was studied by spiking a normal plasma sample with 30 micromolar bromocresol green. That concentration corresponds to roughly 1 equivalent of the compound per HSA protein molecule.

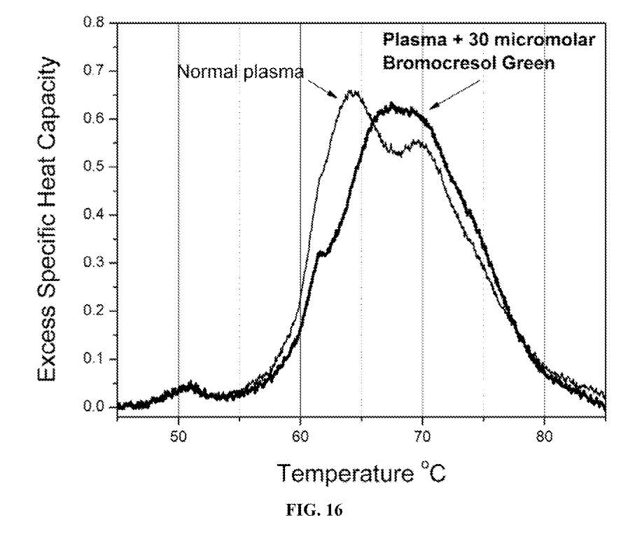

With reference to FIG. 16, the bromocresol green spike causes the plasma thermogram to shift to higher temperatures, in this case because the thermal denaturation of HSA is stabilized by binding of the small molecule. This test shows that addition of small components to plasma can in fact drastically alter the plasma thermogram, even though the actual melting of the added component can not itself be seen. The alteration results from stabilization of one or more of the more abundant components.

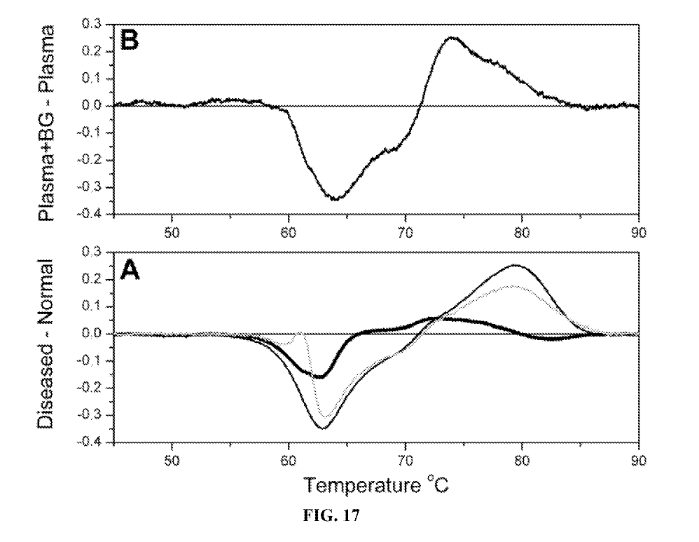

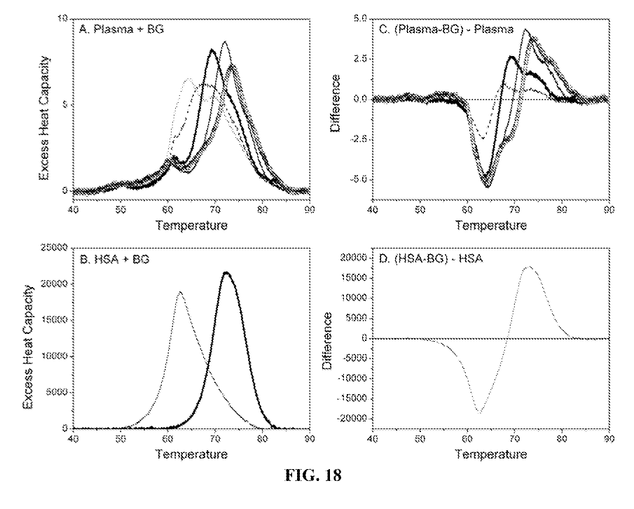

The results of another study are shown in FIG. 17, which indicate that the binding of bromocresol green to HSA within normal plasma mimic the effects of putative biomarker binding. FIG. 17 (Panel A) shows “difference thermograms” for diseased states, obtained by subtracting the normal thermogram from the diseased thermograms seen in FIG. 13. These difference plots feature a negative peak near 62° C., attributable to a shift in HSA denaturation to higher temperatures. Positive difference peaks are evident at 70° C. and higher, attributable to denaturation of ligated HSA (or other proteins). Such behavior can be mimicked by addition of bromocresol green (FIG. 17 (Panel B)). FIG. 17 (Panel B) shows a difference thermogram calculated from normal plasma samples with and without added bromocresol green. (More details of experiments showing the effects of bromocresol green on plasma and pure HSA are shown in FIG. 18). The shape of the difference thermogram is qualitatively similar to those seen for diseased plasma samples, suggesting that the “interactome” hypothesis has merit, and provides a plausible explanation for shifts in thermograms observed in FIG. 13.

The shifts in denaturation transition curves that accompany ligand binding to protein are well understood, and have been explained by a number of specific statistical mechanical and thermodynamic models (Brandts (1990) and Schellman (1958)). The effects of binding on the magnitude and exact shape of a melting transition curve depends precisely on the ligand binding affinity, enthalpy, and stoichiometry. Complex multiphasic transition curves can result from partial saturation. Peptide biomarkers in plasma could produce a myriad of thermogram shapes, depending on the exact proteins (and protein binding sites) that they occupy, and their affinity. The interactions of multiple unique biomarkers with different plasma proteins could produce unique, characteristic thermograms that reflect the underlying complexity of the interactions. While calorimetry may not sense signals arising from the denaturation of the biomarkers themselves, it is uniquely sensitive to interactions of these biomarkers with the more abundant plasma proteins.

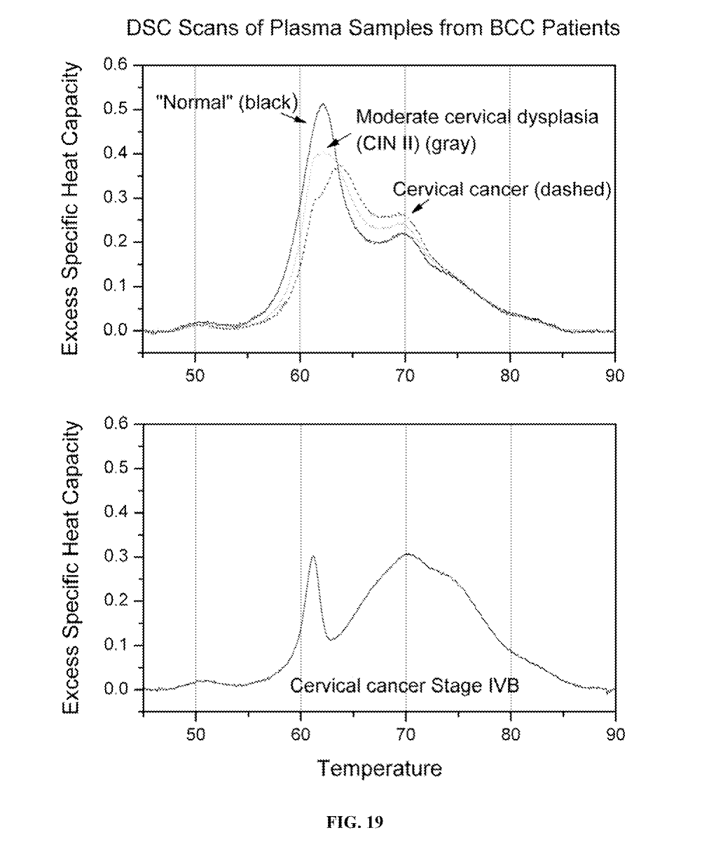

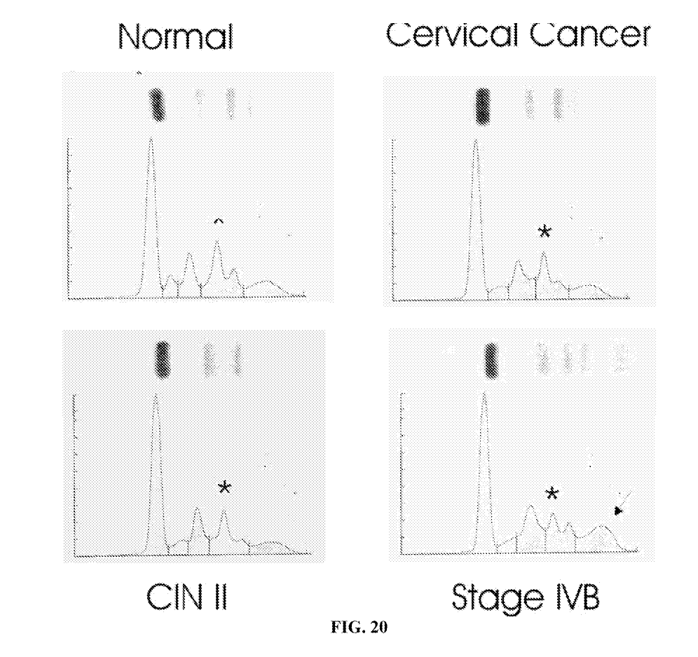

Distinctive thermograms for samples associated with additional conditions of interest. Plasma samples were obtained from subjects diagnosed with cervical cancer (samples obtained from a gynecological cancer tissue bank maintained at the University of Louisville). Thermograms were generated using the cervical cancer samples. The samples were associated with either moderate cervical dysplasia (CIN II), early stage cervical cancer, or stage IVB cervical cancer. With reference to FIG. 19, it was surprisingly found that unique thermograms are generated for particular stages of cervical cancer. As the condition progresses, the thermograms change. Compared to normal plasma, there are distinctive shifts in the thermograms as the disease progresses from moderate cervical dysplasia, through early stage cervical cancer, to the critically ill stage IVB cervical cancer. The changes in the thermograms are unique for each stage, and their patterns are further distinct in detail from the diseased states (lupus, Lyme disease, arthritis) shown in FIG. 13.

Aliquots of these identical samples were also analyzed by the FDA approved serum protein electrophoresis assay. Densitometric scans of the stained gels are shown for comparison in FIG. 20. In comparison to the DSC thermograms, these electrophoretic scans show only subtle changes throughout the progression of the cancer. Standard quantitative analysis of the electrophoresis did not reveal any dramatic systematic changes in the concentrations of protein fractions. This comparison indicates that thermograms reveal differences in plasma that are not readily visible by traditional serum plasma electrophoresis, indicating that the methods of the presently-disclosed subject matter are valuable complements to existing procedures.

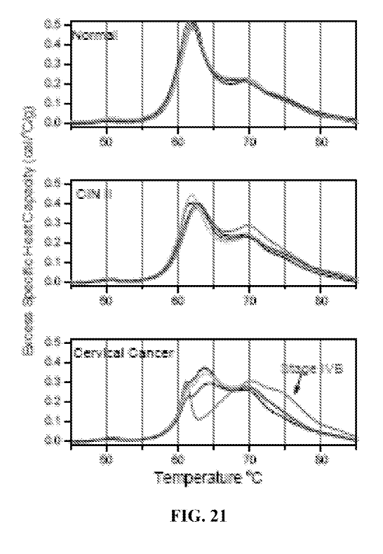

For the cervical cancers, thermograms were generated for several samples from the gynecological tissue bank. Samples from four normals, four CIN II cervical dysplasia, and four diagnosed cervical cancers were studied. These results are plotted in FIG. 21 and depict the reproducibility of the thermograms. The data for the diagnosed cervical cancers clearly show one pronounced outlier. These samples were originally ran blind, using deidentified samples without knowing the exact diagnoses. Upon identification of the outlier thermogram, it was subsequently identified as being from a stage IVB patient, late in progression, and clinically distinct from the other samples that had been provided. This provided an unexpected illustration of the present method's ability to distinguish between particular stages of the disease.

Using methods described herein, thermograms are obtained using plasma samples from normal subjects and from subjects diagnosed with a variety of cancers in order to explore and discover the range of patterns resulting from these diseases. Deidentified plasma samples are obtained from a tissue bank maintained at the University of Louisville. This resource maintains “discard” pieces of benign, premalignant, and malignant gynecological tissues for each patient donor, along with pre- and post-operative blood and urine samples, and ascites fluid (when possible). Plasma is prepared from blood samples by standard methods and was stored at −80° C.

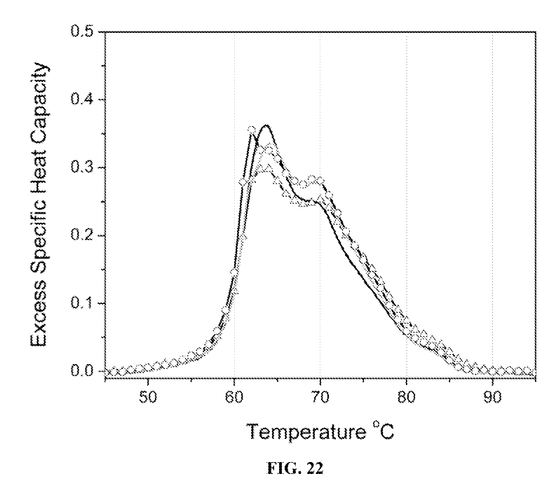

With reference to FIG. 22, thermograms were generated using samples from subjects diagnosed with ovarian cancer, endometrial cancer, and uterine cancer. The solid black line is the average thermogram from 10 normal female subjects; the open triangles show the average thermogram from 12 subjects with ovarian cancer; the solid gray line is the average thermogram from 8 subjects with endometrial cancer; the open circles show the average thermogram from 2 subjects with uterine cancer. These results indicate that ovarian cancer, endometrial cancer, and uterine cancer yield unique thermograms, that are distinct from normal thermograms, distinct from each other, and distinct from the thermograms associated with other conditions, e.g., cervical cancer, arthritis, lupus, Lyme disease.

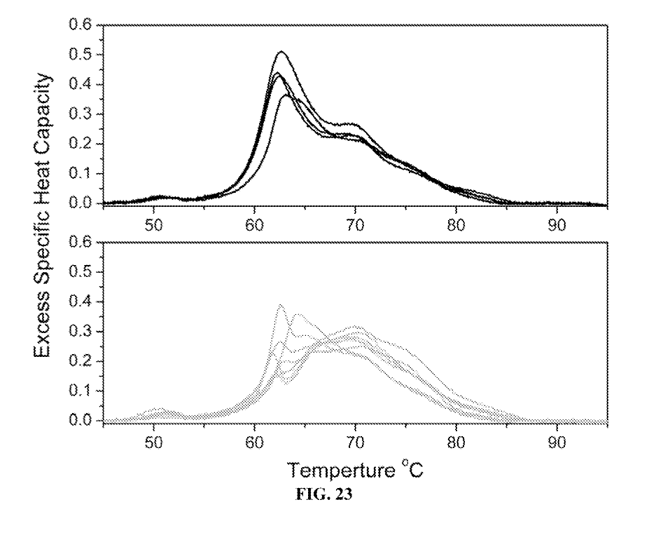

With reference to FIG. 23, thermograms were generated using samples from subjects diagnosed with melanoma. The solid black lines correspond to thermograms of samples obtained from subjects that have undergone successful treatment for melanoma and show no evidence of disease. The solid gray lines correspond to thermograms obtained from subjects with advanced melanoma. These results indicate that different stages of melanoma progression could yield unique thermograms. These results further indicate that melanoma thermograms are distinct from normal thermograms, and distinct from the thermograms associated with other conditions. These results further illustrate that the utility of embodiments of the method of the presently-disclosed subject matter for assessing or monitoring a treatment program, i.e., note the distinction between the thermograms associated with advanced melanoma, and the thermograms associated with successful treatment of melanoma, as well as the trend of the successful treatment thermograms towards a good simulation of a normal thermogram.

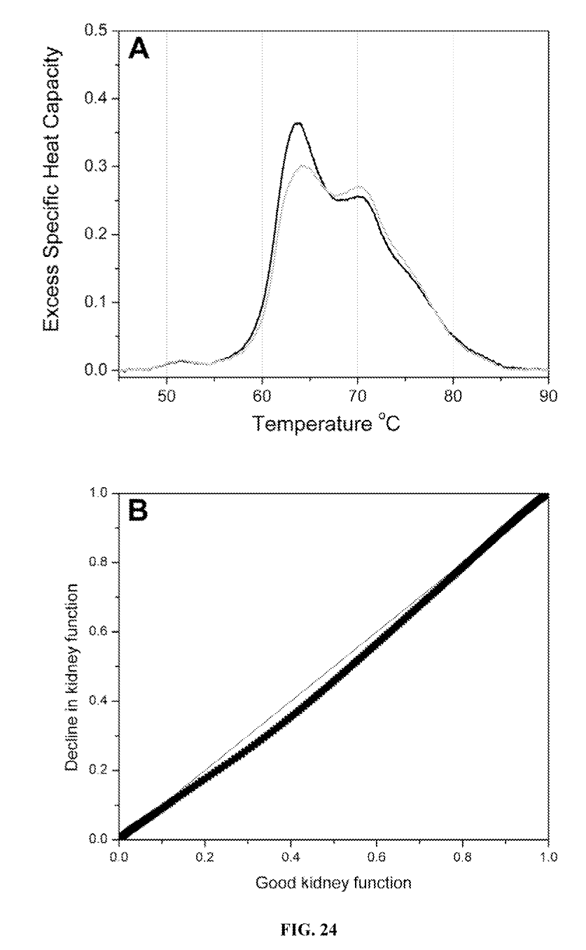

Using methods described herein, thermograms are obtained using plasma samples from normal subjects and from subjects diagnosed with a variety of conditions. With reference to FIG. 24, thermograms were generated using samples obtained prospectively from diabetic subjects exhibiting subsequent differences in future kidney function. Panel A shows average thermograms from two groups of subjects grouped on the basis of kidney function. The solid black line shows an average thermogram from 17 subjects with good kidney function, and the solid gray line is an average thermogram from 15 subjects exhibiting a decline in kidney function. Panel B shows a quantile-quantile plot. This is a graphical technique for determining if two data sets come from populations with a common distribution. If the two sets come from a population with the same distribution they will lie along the 45-degree reference line. The greater the departure from this reference line, the greater the evidence for the conclusion that the two data sets have come from populations with different distributions. Note the deviations from the 45-degree reference line. These results indicate that yet another condition-of-interest yields a unique thermogram.

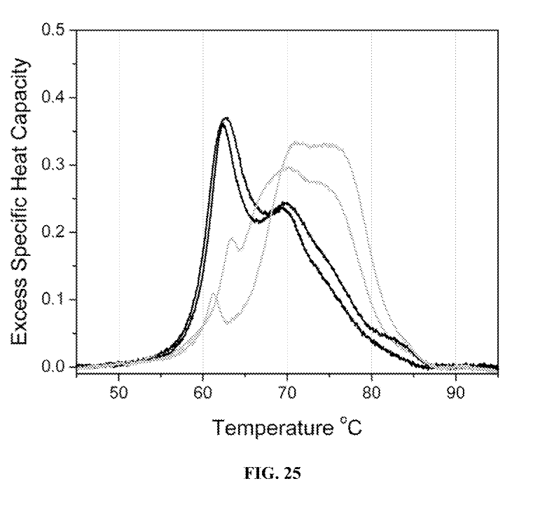

With reference to FIG. 25, thermograms were generated using samples from diabetic subjects with either minimal (CAD−) or severe (CAD+) coronary artery disease. The solid black lines correspond to CAD− patients and the solid gray lines to CAD+ patients. These results provide further evidence that each unique condition can yield a unique thermogram, useful for the methods of the presently-disclosed subject matter.

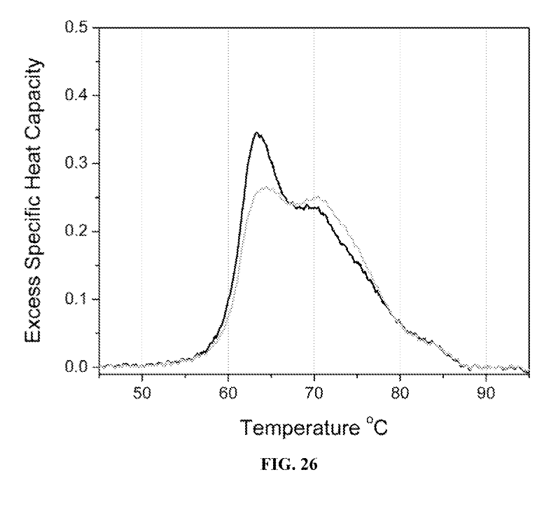

With reference to FIG. 26, thermograms were generated using samples from subjects with amyotrophic lateral sclerosis (ALS). The solid black line corresponds to the average thermogram obtained from 9 normal subjects; the solid gray line corresponds to the average thermogram obtained from 9 subjects with ALS disease. These results provide still further evidence that each unique condition can yield a unique thermogram, useful for the methods of the presently-disclosed subject matter.

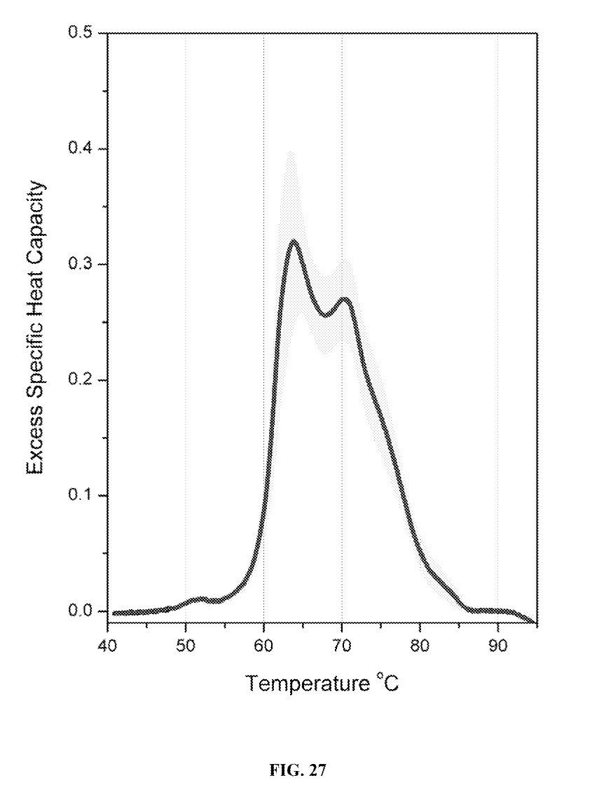

Gender-specific and ethnic group-specific thermograms were studied. With reference to FIG. 27, an average normal thermogram was generated using samples obtained from 100 normal subjects. The subjects were between the ages of 18 and 61, and included: 25 white males, 25 white females, 10 black males, 10 black females, 15 Hispanic males, and 15 Hispanic females. The gray shaded area is the standard deviation for each temperature.

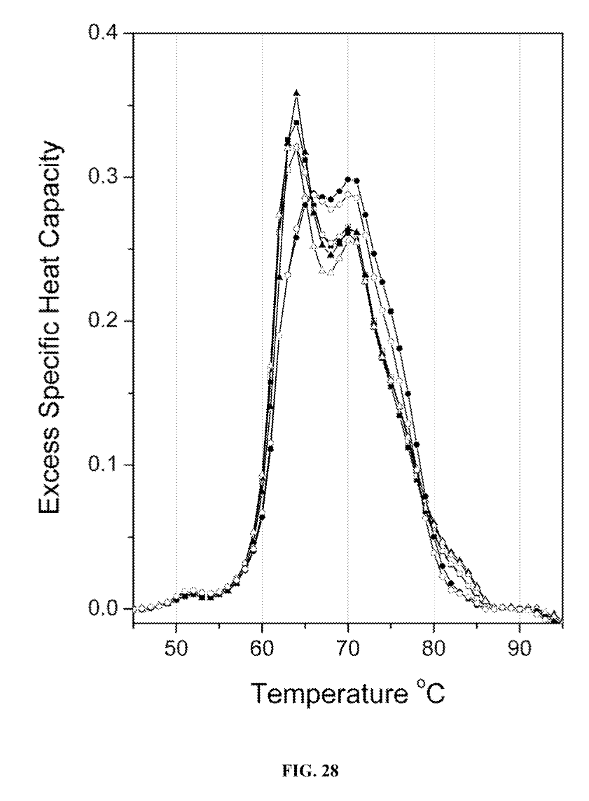

With reference to FIG. 28, the data were separated to generate a series of gender- and ethnic group-specific thermograms. The solid squares represent the average thermogram obtained from 25 white males, the open squares represent the average thermogram obtained from 25 white females, the solid triangles represent the average thermogram obtained from 10 black males, the open triangles represent the average thermogram obtained from 10 black females, the solid circles represent the average thermogram obtained from 15 hispanic males, the open circles represent the average thermogram obtained from 15 hispanic females. It is apparent from inspection of the thermograms that there is a difference in the thermograms of Hispanic subjects, as compared to the thermograms of the other subjects.

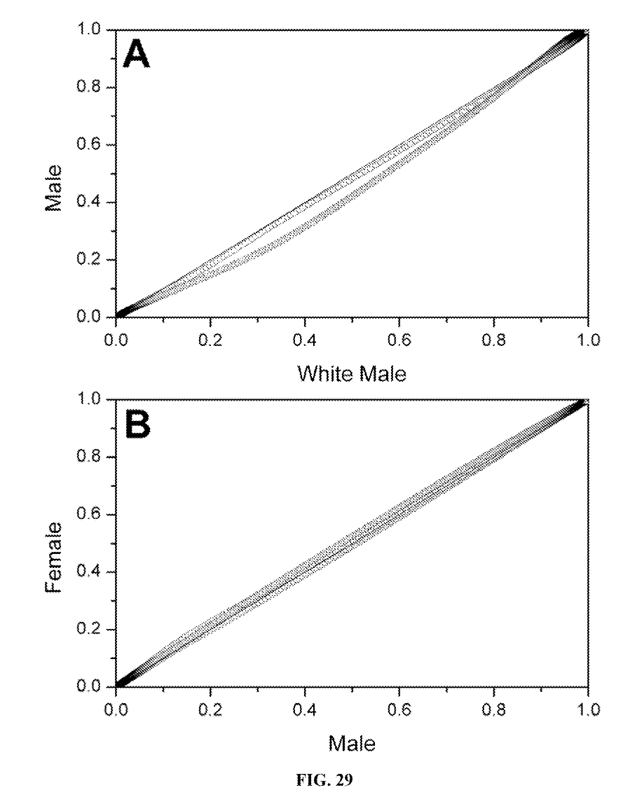

Turning now to FIG. 29, quantile-quantile plots are generated using data presented in FIG. 28. FIG. 29 (Panel A) shows a quantile-quantile plot of the differences between ethnicities. This plot shows the differences in distribution between the average thermograms for white males and males of other ethnicity. The circles represent differences between white and black males and the triangles represent differences between white and hispanic males. It can be seen that the average thermogram for hispanic males is significantly different from that for both white and black males. FIG. 29 (Panel B) shows a quantile-quantile plot of the differences between gender. Here quantile-quantile plots are constructed between males and females of the same ethnicity. The squares represent white subjects, the circles represent black subjects and the triangles represent hispanic subjects. It can be seen that there is negligible differences between genders of the same ethnicity.

The data set forth in FIG. 28 and FIG. 29 indicate that, in some embodiments of the presently-disclosed subject matter, it can be desirable to use Hispanic-specific standard thermograms when a Hispanic subject is involved, i.e., Hispanic subject being monitored, diagnosed, etc. Similarly, in some embodiments, it can be desirable to use non-Hispanic-specific standard thermograms when a non-Hispanic subject is involved.

The results of the studies described herein indicate that the methods of the presently-disclosed subject matter are extremely sensitive to binding interactions between proteins. Changes in low-abundance “biomarkers” of conditions of interest that cannot be detected by known methods such as mass spectroscopy or 2-dimensional electrophoresis can be detected with sensitivity using the methods of the presently-disclosed subject matter.

The methods of the presently-disclosed subject are sensitive not only to changes in protein compositions in a noninteracting mixture, but also to interactions resulting from increased concentrations of smaller components (e.g., “biomarkers”) that would themselves not be directly observed. In either case, reproducible signature changes in thermograms relative to normal samples are seen.

Normal thermograms and thermograms for specific conditions of interest are reproducible and distinct. A thermogram for a specific condition of interest is different than a normal thermogram, and is also different than thermograms for other conditions of interest, i.e., they are poor simulations of one another. Each condition of interest has a distinctive and characteristic thermogram. Indeed, in some embodiments, different stages of a condition of interest have distinctive and characteristic thermograms. Therefore, the methods of the presently-disclosed subject matter have beneficial clinical utility and research utility. Benefits of the methods include, the sensitivity, simplicity, non-invasive sample collection, ability to work with low-volume samples, ease of sample preparation, and the capacity for high-throughput.

Materials and Methods

Pure protein samples. Human serum albumin (HSA) (lot # 113K7601), immunoglobulin G (IGG) (lot # 415781/1), immunoglobulin A (IGA) (lot # 105K3777), α1-acid glycoprotein (AAG) (lot # 073K7607), α1-antitrypsin (AAT) (lot # 033K7603), fibrinogen (FIB) (lot # 083K7604), transferrin (TRF) (lot # 123K14511), haptoglobin (HPT) (lot # 055K1664) and immunoglobulin M (IGM) (lot # 016K4876) were purchased from Sigma-Aldrich Chemical Co. (St. Louis, Mo.). α1-Antichymotrypsin (ACT) (lot # B58700), complement C3 (C3) (lot # D33204), complement C4 (C4) (lot # D34721), ceruloplasmin (CER) (lot # B70322), α2-macroglobulin (A2M) (lot # B73605) and prealbumin (PRE) (lot # B68296) were purchased from Calbiochem. C-reactive protein (CRP) (lot # 32F0305FP) was purchased from Life Diagnostics.

Manufactured mixtures. By using available purified plasma proteins, solution mixtures of any desired composition can be made and thermograms for these preparations can be obtained. Such is done, in order to match experimental thermograms of normal and diseased plasma/serum samples. This approach allows for an exploration of the effects of individual components on thermogram shape.

Standard reference serum. A serum reference material (sample # 16910) was purchased from Sigma-Aldrich Chemical Co. (St. Louis, Mo.). A standardized human serum sample can be provided with a certificate of analysis that includes certified values for the concentrations (g/L) of the 15 most abundant proteins, along with the uncertainty in the concentration determination. Concentrations of each sample are determined on the same sample independently by multiple different laboratories. Each sample is provided as a lyophilized portion under nitrogen, and a strict standardized protocol for reconstitution of the material is provided. Thermograms obtained for such materials are useful for multicomponent analysis, since the protein concentrations that are being sought by the numerical analyses procedure are precisely known for the experimental sample. The goodness of fits can thus be rigorously evaluated.

Plasma samples. Normal plasma samples (lot # JA053759, JA053761, JA053763, JA053764, JA053765, JA053766, JC014372, JM034968, JM034969, JM034970, JM034971) were purchased from Innovative Research (Southfield, Mich.) and were also obtained from the Gynecological Cancer Repository of the James Graham Brown Cancer Center. Plasma from subjects suffering from Lyme disease (lot # BM146897, BM140032, BM140031, BM140028), systemic lupus erythematosis (lot # BM142168, BM142160) and rheumatoid arthritis (lot # BM204810, BM205222, BM203373, BM202803, BM200182) were purchased from BBI Diagnostics (West Bridgewater, Mass.).

Sample preparation. IGM, C3, C4 and CRP were purchased as solutions in buffer, lyophilized to dryness and then re-constituted in a smaller volume of ultrapure water (18.2 MΩ-cm) to yield a concentration suitable for DSC. PRE, A2M, CER, ACT were purchased as a powder lyophilized from buffer and were reconstituted with ultrapure water. HSA, IGG, IGA, AAG, AAT, FIB, TRF and HPT were reconstituted with 10 mM potassium phosphate, 150 mM NaCl, pH 7.5. Reference serum was reconstituted according to the guidelines. Pure proteins and reference serum were dialyzed for 24 h at 4° C. against 10 mM potassium phosphate, 150 mM NaCl, pH 7.5 to ensure complete solvent exchange. Pure proteins were diluted with dialysate to a concentration suitable for DSC. Reference serum was diluted 25-fold with the dialysate. Plasma samples (100 μL) were dialyzed for 24 h at 4° C. against 10 mM potassium phosphate, 150 mM NaCl, 0.38% (w/v) sodium citrate, pH 7.5 to ensure complete solvent exchange then diluted 25-fold with the same buffer. All samples (0.45 micron, cellulose acetate or polyethersulfone) and buffers (0.22 micron, polyethersulfone) were filtered before use. Pure protein concentrations were quantitated spectrophotometrically using the following extinction coefficients (ε280; L−1·g−1·cm −1): HSA, 0.53; IGG, 1.38; IGA, 1.32; AAG, 0.89; AAT, 0.53; FIB, 1.55; TRF, 1.12; HPT, 1.2; IGM, 1.18; ACT, 0.62; C3, 0.97; C4, 0.92; CER, 1.49; A2M, 0.893; PRE, 1.41; CRP, 1.95.

DSC protocol. An automated capillary Differential Scanning Calorimeter (DSC) (MicroCal, LLC, Northampton, Mass.) was used for the studies described herein. Samples and dialysate were stored in 96-well plates at 5° C. until being loaded into the calorimeter using the robotic attachment. Scans were recorded from 20-110° C. at 1° C./min using the mid feedback mode, a filtering period of 2 s and with a pre-scan thermostat of 15 min. Data were analyzed using Origin 7.0. Sample scans were first corrected for the instrument baseline by subtracting an appropriate buffer scan. Nonzero baselines were then corrected by applying a linear baseline fit. Scans were finally normalized for the gram concentration of protein. For the pure protein samples, protein concentrations were determined spectrophotometrically as outlined herein. Total protein concentrations of the reference serum and plasma samples were measured by the bicinchoninic acid method (Pierce, Rockford, Ill.). Thermograms were plotted as Excess Specific Heat Capacity (cal/° C. g) versus temperature.

Clinical Laboratory Testing. Both total protein and the concentration of the individual major serum proteins are measured, for example, immunoglobulins G, A and M, transferrin, haptoglobin, prealbumin, complement factors C3 and C4, ceruloplasmin, apolipoproteins A1 and B, α1-antitrypsin, α1-acid glycoprotein, and C-reactive protein. In addition, serum (or plasma) protein electrophoresis is performed on each sample. All of these assays are performed by FDA approved, standard clinical laboratory procedures. The concentrations of the specific serum proteins and the SPE patterns are correlated with the thermograms determined by the methods described herein.

Lipoproteins (HDL, LDL, VLDL, and chylomicrons) are more complex than the other serum proteins. They contain not only the apolipoproteins, but also cholesterol and triglyceride, as well as other minor components. The lipoproteins are likely to cause a significant signal in the thermogram patterns. Therefore, cholesterol and triglyceride of the samples are also measured. Cholesterol and triglyceride is measured on the Vitros by enzymatic methods.

C-reactive protein (CRP) is normally present at a low concentration, which is unlikely to contribute to the thermogram pattern. However, during the acute phase reaction, which is common among sick patients, the concentration of CRP can be high enough to be detectable by the methods described herein.

Clinical assay methods. Protein electrophoresis was performed on agarose gels using the SPIFE 3000 and scanned with the QUICKSCAN 2000 (Helena Laboratories, Beaumont, Tex.). Total protein was measured by the biuret method on the Ortho Vitros 950 (Vitros) (Ortho-Clinical Diagnostic, Rochester, N.Y.) chemistry analyzer. Albumin was measured on the Vitros by the bromocresol green dye binding assay or by an immunoturbidometric assay on the Cobas Integra 800 (Integra) (Roche, Indianapolis, Ind.). Albumin concentrations were also determined from the fraction percent on the protein electrophoresis assay along with the total protein concentration. Specific serum proteins (IGG, IGA, TRF, HPT, IGM, C3, C4, PRE, CRP) were measured by immunoturbidimetry on the Integra.

Column depletion experiments. Reference serum was depleted of HSA using the SwellGel Blue™ albumin removal kit with some minor modifications to the manufacturer's protocol (Pierce, Rockford, Ill.). The serum sample was diluted 10-fold into 10 mM potassium phosphate, pH 7.5 in order to achieve salt conditions and albumin concentrations required for good column binding. Diluted serum (200 μL) was applied to a column containing 2 Swellgel™ discs. An HSA-depleted fraction was obtained following the standard protocol. A single 200 μL volume of the supplied binding/wash buffer was used to obtain a wash fraction. Finally, an eluted HSA fraction was obtained from a single 200 μL addition of the supplied elution buffer. In order to obtain a greater volume of each fraction for subsequent experiments, multiple columns were run using an identical protocol and each of the fractions pooled. Fractions for DSC analysis were dialyzed for 24 h at 4° C. against 10 mM potassium phosphate, 150 mM NaCl, pH 7.5 and diluted as necessary with dialysate. DSC scans were performed on an N-DSC II instrument (Calorimetry Sciences Corporation, Provo, Utah) from 20-110° C. at 1° C./min with a pre-scan equilibration time of 10 min. Data were analyzed using Origin 7.0.

### REFERENCES

Throughout this document, various references are mentioned. All such references are incorporated herein by reference, including the references set forth in the following list:
1. Anderson, N. L. and N. G. Anderson, The human plasma proteome: history, character, and diagnostic prospects. Mol Cell Proteomics, 2002. 1(11): p. 845-67.
2. Anderson, N. L., M. Polanski, R. Pieper, T. Gatlin, R. S. Tirumalai, T. P. Conrads, T. D. Veenstra, J. N. Adkins, J. G. Pounds, R. Fagan, and A. Lobley, The human plasma proteome: a nonredundant list developed by combination of four separate sources. Mol Cell Proteomics, 2004. 3(4): p. 311-26.
3. Omenn, G. S., D. J. States, M. Adamski, T. W. Blackwell, R. Menon, H. Hermjakob, R. Apweiler, B. B. Haab, R. J. Simpson, J. S. Eddes, E. A. Kapp, R. L. Moritz, D. W. Chan, A. J. Rai, A. Admon, R. Aebersold, J. Eng, W. S. Hancock, S. A. Hefta, H. Meyer, Y. K. Paik, J. S. Yoo, P. Ping, J. Pounds, J. Adkins, X. Qian, R. Wang, V. Wasinger, C. Y. Wu, X. Zhao, R. Zeng, A. Archakov, A. Tsugita, I. Beer, A. Pandey, M. Pisano, P. Andrews, H. Tammen, D. W. Speicher, and S. M. Hanash, Overview of the HUPO Plasma Proteome Project: results from the pilot phase with 35 collaborating laboratories and multiple analytical groups, generating a core dataset of 3020 proteins and a publicly-available database. Proteomics, 2005. 5(13): p. 3226-45.
4. Aebersold, R., L. Anderson, R. Caprioli, B. Druker, L. Hartwell, and R. Smith, Perspective: a program to improve protein biomarker discovery for cancer. J Proteome Res, 2005. 4(4): p. 1104-9.
5. Anderson, N. L., The roles of multiple proteomic platforms in a pipeline for new diagnostics. Mol Cell Proteomics, 2005. 4(10): p. 1441-4.
6. Ebert, M. P., M. Korc, P. Malfertheiner, and C. Rocken, Advances, challenges, and limitations in serumproteome-based cancer diagnosis. J Proteome Res, 2006. 5(1): p. 19-25.
7. Mor, G., I. Visintin, Y. Lai, H. Zhao, P. Schwartz, T. Rutherford, L. Yue, P. Bray-Ward, and D. C. Ward, Serum protein markers for early detection of ovarian cancer. Proc Natl Acad Sci USA, 2005. 102(21): p. 7677-82.
8. Rosenblatt, K. P., P. Bryant-Greenwood, J. K. Killian, A. Mehta, D. Geho, V. Espina, E. F. Petricoin, 3rd, and L. A. Liotta, Serum proteomics in cancer diagnosis and management. Annu Rev Med, 2004. 55: p. 97-112.
9. Wulfkuhle, J. D., L. A. Liotta, and E. F. Petricoin, Proteomic applications for the early detection of cancer. Nat Rev Cancer, 2003. 3(4): p. 267-75.
10. Wulfkuhle, J. D., C. P. Paweletz, P. S. Steeg, E. F. Petricoin, 3rd, and L. Liotta, Proteomic approaches to the diagnosis, treatment, and monitoring of cancer. Adv Exp Med Biol, 2003. 532: p. 59-68.
11. O'Connell, T. X., T. J. Horita, and B. Kasravi, Understanding and interpreting serum protein electrophoresis. Am Fam Physician, 2005. 71(1): p. 105-12.
12. Craig, W., T. Ledue, and R. Ritchie, Plasma Proteins: Clinical Utility and Interpretation. 2004, Scarborough, Me.: Foundation for Blood Research. 152.
13. Anderson, L. and N. G. Anderson, High resolution two-dimensional electrophoresis of human plasma proteins. Proc Natl Acad Sci USA, 1977. 74(12): p. 5421-5.
14. Anderson, N. L. and N. G. Anderson, A two-dimensional gel database of human plasma proteins. Electrophoresis, 1991. 12(11): p. 883-906.
15. Gygi, S. P. and R. Aebersold, Mass spectrometry and proteomics. Curr Opin Chem Biol, 2000. 4(5): p. 489-94.
16. Liotta, L. A., E. C. Kohn, and E. F. Petricoin, Clinical proteomics: personalized molecular medicine. JAMA, 2001. 286(18): p. 2211-4.
17. Yates, J. R., 3rd, Mass spectrometry. From genomics to proteomics. Trends Genet, 2000. 16(1): p. 5-8.
18. Adkins, J. N., S. M. Varnum, K. J. Auberry, R. J. Moore, N. H. Angell, R. D. Smith, D. L. Springer, and J. G. Pounds, Toward a human blood serum proteome: analysis by multidimensional separation coupled with mass spectrometry. Mol Cell Proteomics, 2002. 1(12): p. 947-55.
19. Liotta, L. A. and E. F. Petricoin, Serum peptidome for cancer detection: spinning biologic trash into diagnostic gold. J Clin Invest, 2006. 116(1): p. 26-30.
20. Liotta, L. A., M. Ferrari, and E. Petricoin, Clinical proteomics: written in blood. Nature, 2003. 425(6961): p. 905.
21. Zhou, M., D. A. Lucas, K. C. Chan, H. J. Issaq, E. F. Petricoin, 3rd, L. A. Liotta, T. D. Veenstra, and T. P. Conrads, An investigation into the human serum “interactome”. Electrophoresis, 2004. 25(9): p. 1289-98.
22. Fenby, D. V., Heat: its measurement from Balileo to Lavosier. Pure Applied Chemistry. 1987. 59: p. 91-1000.
23. Biltonen, R. L. and E. Freire, Thermodynamic characterization of conformational states of biological macromolecules using differential scanning calorimetry. CRC Crit Rev Biochem, 1978. 5(2): p. 85-124.
24. Brandts, J. F. and L. N. Lin, Study of strong to ultratight protein interactions using differential scanning calorimetry. Biochemistry, 1990. 29(29): p. 6927-40.
25. Bruylants, G., J. Wouters, and C. Michaux, Differential scanning calorimetry in life science: thermodynamics, stability, molecular recognition and application in drug design. Curr Med Chem, 2005. 12(17): p. 2011-20.
26. Freire, E., Differential scanning calorimetry. Methods Mol Biol, 1995. 40: p. 191-218.
27. Sanchez-Ruiz, J. M., Differential scanning calorimetry of proteins. Subcell Biochem, 1995. 24: p. 133-76.
28. Schellman, J. A., The factors affecting the stability of hydrogen-bonded polypeptide structures in solution. Journal of Physical Chemistry, 1958. 62: p. 1485-1494.
29. Peters, T., Jr., All About Albumin: Biochemistry, Genetics and Medical Applications. 1996, San Diego Academic Press.
30. Doumas, B. T., D. D. Bayse, R. J. Carter, T. Peters, Jr., and R. Schaffer, A candidate reference method for determination of total protein in serum. I. Development and validation. Clin Chem, 1981. 27(10): p. 1642-50.
31. Corcoran, R. M. and S. M. Durnan, Albumin determination by a modified bromcresol green method. Clin Chem, 1977. 23(4): p. 765-6.
32. Allain, C. C., L. S. Poon, C. S. Chan, W. Richmond, and P. C. Fu, Enzymatic determination of total serum cholesterol. Clin Chem, 1974. 20(4): p. 470-5.
33. Cooper, G. R., G. L. Myers, S. J. Smith, and E. J. Sampson, Standardization of lipid, lipoprotein, and apolipoprotein measurements. Clin Chem, 1988. 34(8B): p. B95-105.
34. Eda, S., J. Kaufmann, W. Roos, and S. Pohl, Development of a new microparticle-enhanced turbidimetric assay for C-reactive protein with superior features in analytical sensitivity and dynamic range. J Clin Lab Anal, 1998. 12(3): p. 137-44.
35. Gorman, L. S. and D. Adkins, Apoprotein assays: radial immunodiffusion versus an immunoturbidometric technique. Clin Lab Sci, 1991. 4(2): p. 110-2.
36. Larkin, T. C., T. A. Fingleton, M. McCusker, M. Cassidy, G. Gunzer, C. A. Lepp, and E. Gamboa, A new Olympus assay for the determination of ceruloplasmin. Clin Lab, 2004. 50(3-4): p. 193-203.
37. Levinson, S. S. and S. G. Wagner, Immunonephelometric/turbidimetric apolipoprotein B assays for the clinical laboratory. Clin Chim Acta, 1993. 223(1-2): p. 31-42.
38. Lolekha, P. H., A. Chittamma, W. L. Roberts, P. Sritara, S. Cheepudomwit, and P. Suriyawongpaisal, Comparative study of two automated high-sensitivity C-reactive protein methods in a large population. Clin Biochem, 2005. 38(1): p. 31-5.
39. Molinari, E. A. P. F. Pichler, H. Grillhofer, and G. M. Kostner, Immunoturbidimetric determination of lipoprotein(a): improvement in the measurement of turbid and triglyceride-rich samples. Clin Chim Acta, 1995. 235(1): p. 59-69.
40. Sakurabayashi, I., Y. Saito, T. Kita, Y. Matsuzawa, and Y. Goto, Reference intervals for serum apolipoproteins A-I, A-II, B, C-II, C-III, and E in healthy Japanese determined with a commercial immunoturbidimetric assay and effects of sex, age, smoking, drinking, and Lp(a) level. Clin Chim Acta, 2001. 312(1-2): p. 87-95.
41. Sanchez, A., J. L. Mirabel, E. Barrenechea, J. Eugui, A. Puelles, and A. Castaneda, Evaluation of an improved immunoturbidimetic assay for serum C-reactive protein on a COBAS INTEGRA 400 Analyzer. Clin Lab, 2002. 48(5-6): p. 313-7.
42. Howerton, D. A., I. J. Check, and R. L. Hunter, Densitometric quantitation of high resolution agarose gel protein electrophoresis. Am J Clin Pathol, 1986. 85(2): p. 213-8.
43. Cantor, C. and P. Schimmel, Biophysical Chemistry Part 1: The conformation of biological macromolecules. 1980, San Francisco: W.H. Freeman. page 37.
44. Lodder, R. A. and Hieftje, G. M., Quantile analysis: a method for characterizing data distributions. Applied Spectroscopy, 1988. 42: 1512-1520.
45. Young, I. T., Proof without prejudice: use of the Kolmogorov-Smirnov test for the analysis of histograms from flow systems and other sources. Journal of Histochemistry and Cytochemistry, 1977. 25: 935-941.
46. Motulsky, H. Intuitive Biostatisitics, (Oxford University Press, New York, 1995).
47. Garbett, N. C., Miller, J. J., Jenson, A. B., Miller, D. M., Chaires, J. B., Interrogation of the Plasma Proteome with Differential Scanning Calorimetry. Clinical Chemistry, 2007. 53: 2012-2014.
48. Garbett, N. C., Miller, J. J., Jenson, A. B., Chaires, J. B., Calorimetric Analyisis of the Plasma Proteome. Seminars in Nephrology 27(6): 621-6.
49. Garbett, N. C., Miller, J. J., Jenson, A. B., Chaires, J. B., Ligand Binding Alters the Calorimetric Thermogram of Albumin. J. Clinical Ligand Assay, 2006*. 29:4, 1-4.(*Article written and submitted in 2007, notwithstanding Winter 2006 identification of J. of Clinical Ligand Assay publication).
50. Garbett, N. C., Miller, J. J., Jenson, A. B., Chaires, J. B. (2008) Calorimetry outside the box: a new window into the plasma proteome. Biophysical Journal 94: in press.

What is claimed is:

1. A method of diagnosing or monitoring a condition of interest in a subject, comprising:
generating a signature thermogram containing a protein composition pattern for a sample obtained from the subject; and
comparing the signature thermogram to a standard thermogram selected from:
a negative standard thermogram containing a protein composition pattern associated with an absence of the condition of interest; and
a positive standard thermogram containing a protein composition pattern associated with a presence of the condition of interest; and
identifying the subject as having the condition of interest or lacking the condition of interest.

2. The method of claim 1, further comprising: identifying the subject as having the condition of interest when the signature thermogram is a good simulation of the positive standard thermogram.

3. The method of claim 2, further comprising: identifying the subject as having the condition of interest when the signature thermogram is a good simulation of the positive standard thermogram, and the signature thermogram is a poor simulation of the negative standard thermogram.

4. The method of claim 1, further comprising: identifying the subject as lacking the condition of interest when the signature thermogram is a poor simulation of the positive standard thermogram.

5. The method of claim 1, further comprising: identifying the subject as lacking the condition of interest when the signature thermogram is a good simulation of the negative standard thermogram.

6. The method of claim 5, further comprising: identifying the subject as lacking the condition of interest when the signature thermogram is a poor simulation of the positive standard thermogram, and the signature thermogram is a good simulation of the negative standard thermogram.

7. The method of claim 1, wherein each standard thermogram is a group-specific standard thermogram.

8. The method of claim 7, wherein each group-specific standard thermogram is an ethnic group-specific standard thermogram.

9. The method of claim 8, wherein each ethnic group-specific standard thermogram is: a Hispanic-specific standard thermogram if the subject is Hispanic; or a non-Hispanic-specific standard thermogram if the subject is non-Hispanic.

10. The method of claim 1, wherein the condition of interest is cancer.

11. The method of claim 10, wherein the cancer is selected from: cervical cancer, endometrial cancer, lung cancer, melanoma, multiple myeloma, ovarian cancer, and vulvar cancer.

12. The method of claim 10, wherein the condition of interest is a stage of cervical cancer selected from: moderate cervical dysplasia (CIN II), early stage cervical cancer, and stage IVB cervical cancer.

13. The method of claim 1, wherein the condition of interest is an autoimmune disease.

14. The method of claim 13, wherein the autoimmune disease is selected from: rheumatoid arthritis, multiple sclerosis, and systemic lupus.

15. The method of claim 1, wherein the condition of interest is caused by a bacterial infection.

16. The method of claim 15, wherein the condition is Lyme disease.

17. The method of claim 1, wherein the condition of interest is caused by a viral infection.

18. The method of claim 17, wherein the condition is selected from: Dengue fever, and hepatitis.

19. The method of claim 1, wherein the condition of interest is selected from: amyotrophic lateral sclerosis (ALS), anemia, cardiac disease, diabetes, and renal disease.

20. The method of claim 1, further comprising comparing the signature thermogram to multiple positive standard thermograms, and identifying the subject as having the condition associated with the positive standard thermogram of which the signature thermogram is a good simulation.

21. The method of claim 20, wherein one of the positive standard thermograms is associated with multiple sclerosis, and another of the positive standard thermograms is associated with amyotrophic lateral sclerosis (ALS).

22. The method of claim 20, wherein the multiple positive standard thermograms include positive standard thermograms for different stages of a condition of interest.

23. The method of claim 1, further comprising:
providing a second sample obtained from the subject at a time point after the first sample is obtained;
generating a second signature thermogram containing a protein composition pattern for the second sample;
comparing the first signature thermogram to the second signature thermogram; and
identifying the condition of interest as changed when the second signature thermogram is a poor simulation of the first signature thermogram, or identifying condition of interest as being unchanged when the second signature thermogram is a good simulation of the first signature thermogram.

24. The method of claim 23, further comprising comparing the second signature thermogram to the negative standard thermogram, and identifying the subject as lacking the condition of interest if the second signature thermogram is a good simulation of the negative standard thermogram.

25. The method of claim 23, further comprising comparing the second signature thermogram to positive standard thermograms for different stages of a condition of interest, and identifying the condition as progressing, unchanged, or regressing in the subject.

26. The method of claim 1, wherein the sample is a plasma sample or a serum sample.

27. A method of assessing a treatment program for a subject, comprising:
providing a first sample obtained from the subject at a first time point of interest;
generating a first signature thermogram containing a protein composition pattern for the first sample;
providing a second sample obtained from the subject at a second time point of interest;
generating a second signature thermogram containing a protein composition pattern for the second sample;
comparing the first signature thermogram to the second signature thermogram; and
identifying the presence or absence of a change in the condition of interest.

28. The method of claim 27, further comprising identifying the absence of a change in the condition of interest when the second signature thermogram is a good simulation of the first signature thermogram.

29. The method of claim 27, further comprising identifying the presence of a change in the condition of interest when the second signature thermogram is a poor simulation of the first signature thermogram.

30. The method of claim 27, wherein the first time point of interest occurs prior to the initiation of the treatment program, and the second time point of interest occurs following the initiation of the treatment program.

31. The method of claim 30, and further comprising:
comparing the second signature thermogram to a standard thermogram selected from:
a negative standard thermogram containing a protein composition pattern associated with an absence of the condition of interest; and
a positive standard thermogram containing a protein composition pattern associated with a presence of the condition of interest.

32. The method of claim 27, wherein the samples are plasma samples or serum samples.

33. A method of screening for a composition useful for treating a condition of interest, comprising:
administering to a subject infected with the condition of interest a candidate treatment composition;
providing a sample obtained from the subject;
generating a signature thermogram containing a protein composition pattern for the sample;
comparing the signature thermogram to a standard thermogram selected from:
a negative standard thermogram containing a protein composition pattern associated with an absence of the condition of interest; and
a positive standard thermogram containing a protein composition pattern associated with a presence of the condition of interest; and
determining the utility of the candidate treatment composition.

34. A method of screening a composition for plasma protein interactions, comprising:
interacting the composition with a first plasma sample;
generating a first signature thermogram containing a protein composition pattern for the first plasma sample;
comparing the first signature thermogram to
a negative standard thermogram containing a protein composition pattern associated with an absence of plasma protein interactions; or
a second signature thermogram generated using a second plasma sample not interacted with the composition; and
identifying the composition as lacking substantial plasma protein interactions when the first signature thermogram is a good simulation of
the negative standard thermogram, or
the second signature thermogram.
